(19) United States
(12) Patent Application Publication
(10) Pub. No.: US 2024/0108743 A1
(43) Pub. Date: Apr. 4, 2024

(54) LINKER PEPTIDE FOR CONSTRUCTING FUSION PROTEIN

(71) Applicant: AMPSOURCE BIOPHARMA SHANGHAI INC., Shanghai (CN)

(72) Inventors: Qiang LI, Shanghai (CN); Yuanli LI, Shanghai (CN); Si CHEN, Shanghai (CN); Zhu WANG, Shanghai (CN); Zhao DONG, Shanghai (CN); Zirui LI, Shanghai (CN); Xinlu MA, Shanghai (CN); Lu YANG, Shanghai (CN); Yongjuan GAO, Shanghai (CN); Yuncheng ZHENG, Shanghai (CN); Naichao SUN, Shanghai (CN)

(21) Appl. No.: 18/492,958

(22) Filed: Oct. 24, 2023

Related U.S. Application Data
(62) Division of application No. 17/391,535, filed on Aug. 2, 2021, now Pat. No. 11,833,212.
Application No. 17/391,535 is a continuation of application No. 16/326,412, filed on Feb. 19, 2019, now Pat. No. 11,123,438.

(30) Foreign Application Priority Data
Aug. 19, 2016 (CN) 201610692679.4
Aug. 19, 2016 (CN) 201610694914.1

(51) Int. Cl. A61K 47/65; A61P 3/04; A61P 3/10; C07K 14/475; C07K 14/54; C07K 14/755; C07K 16/06; C07K 16/28; C07K 16/46; C07K 19/00; C12N 15/62; C12N 15/63; C12N 15/85

(52) U.S. Cl. CPC A61K 47/65; A61P 3/04; A61P 3/10; C07K 14/475; C07K 14/5418; C07K 14/755; C07K 16/065; C07K 16/2809; C07K 16/2887; C07K 16/468; C07K 19/00; C12N 15/62; C12N 15/63; C12N 15/85

(57) ABSTRACT

A linker peptide for constructing a fusion protein. The linker peptide comprises a flexible peptide and a rigid peptide. The flexible peptide consists of one or more flexible units. The rigid peptide consists of one or more rigid units. The flexible unit comprises two or more amino acid residues selected from Gly, Ser, Ala, and Thr. The rigid unit comprises a human chorionic gonadotropin β-subunit carboxy-terminal peptide (CTP) bearing a plurality of glycosylation sites. The linker peptide can more effectively eliminate mutual steric hindrance of two fusion molecules, decreasing a reduction/loss of polymerization or activity resulting from improper folding of an active protein or a conformational change. On the other hand, the negatively charged, highly sialylated CTP can resist renal clearance, further prolonging a half-life of a fused molecule and enhancing bioavailability of a fused protein. Furthermore, a protective effect of a glycosylated side chain CTP can lower the protease sensitivity of the linker peptide, making a linker region of the fusion protein less degradable.

### CROSS-REFERENCE TO RELATED APPLICATIONS

This application is a division of application Ser. No. 17/391,535, filed on Aug. 2, 2021, which is a continuation of application Ser. No. 16/326,412, filed on Feb. 19, 2019, now U.S. Pat. No. 11,123,438, which is a United States national stage entry under 35 U.S.C. § 371 of International Application No. PCT/CN2016/106011, filed on Nov. 16, 2016, which claims priority to Chinese Patent Application Nos. 201610692679.4, filed on Aug. 19, 2016, and 201610694914.1, filed on Aug. 19, 2016. The contents of all aforementioned applications are incorporated herein by reference in their entireties.

### SEQUENCE LISTING

The XML-format Sequence Listing submitted herewith, in the file named R1SL15079-0002-02000.xml with a size of 66,221 bytes, was created on December 23, 2023, and is incorporated by reference in its entirety.

### FIELD OF THE INVENTION

The present invention relates to the field of fusion proteins and, more specifically, to a peptide linker for the construction of fusion proteins.

### BACKGROUND

In recent two decades, protein fusion technology has been widely used in the construction of bifunctional antibodies, bifunctional enzymes, and bifunctional proteins. However, a variety of problems have been encountered in the construction of fusion proteins. For example, proteins that fold correctly during expression alone do not fold properly in the fusion protein; the active site is blocked after fusion due to the short distance between the two fused proteins; the fusion protein molecule is easily degraded by proteases when it cannot fold properly or when its conformation has changed; the protein catalytic domain with certain flexibility loses its original function after fusion; and so on. The emergence of these problems often leads to reduction or even complete loss of the activity of the fusion proteins. It is generally believed that the activity of the original protein molecule will decrease to a certain extent after the protein molecule is constructed in the fusion protein. A favorable fusion protein is the one that keeps more than 50% activity of the original protein molecule(s). In order to solve the above problems, researchers conducted many studies and explorations on the design and construction of fusion proteins to improve the activity of fusion proteins. such as changing the linking order of the fused proteins, changing different fusion sites, using different fusion partners, or using a peptide linker, etc.

Compared with other fusion strategies, the use of a peptide linker has a variety of advantages. First, the amino acids that make up the peptide linker are diverse (20 common amino acids). The length of the peptide linker is also an important tunable parameter, which can lead to rich diversity of peptide linkers (20ⁿ, n is the number of amino acid residues of the peptide linker). The peptide linkers are easy for bioengineering modifications. Second, the peptide linker provides certain spatial spacing, such that the two fused proteins fold correctly without interfering with each other. Third, the peptide linker can also provide more interaction possibilities for two fused proteins, promoting synergic interactions.

There are currently two kinds of commonly used peptide linkers, helical rigid peptide linkers (such as A(EAAAK)ₙA (SEQ ID NO: 34)) and flexible peptide linkers comprising less hydrophobic and less charged amino acids. The helical rigid peptide linkers can effectively separate different functional regions of the fusion proteins. Examples of flexible peptide linkers include the (GGGGS)₃ sequence (SEQ ID NO: 35) that Huston designed. In addition, it has been reported that the carboxyl terminal peptide (hereinafter referred to as CTP) of human chorionic gonadotropin (HCG) beta chain can be used as a peptide linker alone, and is mainly used to link different subunits of the same protein. For example, CTP is used as a peptide linker between the beta and alpha subunits of follicle stimulating hormone, as disclosed in Chinese Patent Nos. CN103539860A, CN103539861A, CN103539868A and CN103539869A. In WO2005058953A2, CTP is used as a peptide linker in the fusion protein, linking the glycoprotein beta and alpha subunits. However, because CTP has the effect of prolonging the in vivo half-life of certain proteins, it has been primarily disclosed as a half-life prolonging moiety in the fusion proteins in many other patents. The half-life prolonging moiety can optionally choose CTP, immunoglobulin Fc, or other fusion partners with similar half-life prolonging function.

A number of literatures have reported the effects of the peptide linker sequences on the construction and expression of fusion proteins. For example, in the construction of the single chain antibody 1F7, it was found that using a conventional Genex212 peptide linker (GSTSGSGKSSEGKG (SEQ ID NO: 36)) to link the light and heavy chains did not achieve the original catalytic activity of the protein and the fusion protein was unstable. After a library of 18 amino acid residues with random sequences was constructed and screened, catalytically active single chain antibodies were obtained (Tang Y et al., 1996, J Biol Chem., 271:15682-15686). In studying the helical peptide, Arai et al. found that the fluorescence resonance energy transfer from EBFP to EGFP reduced as the length of the peptide linker increased, suggesting that increasing the linker length could effectively separate the two functional regions (Arai R. et al., 2001, Protein Eng., 14:529-532). In addition, in studying dengue virus NS2B protein, Luo et al. found that inserting a glycine residue between the original sites 173 and 174, or replacing proline 174 with glycine, resulted in a significant decrease in protein activity of both the N- and C-terminal proteins. This indicated that the length and rigidity of the native peptide linker were the result of long-term evolution, and were of great significance to the function of the natural fusion proteins (Luo D. et al., 2010, J Biol Chem., 285:18817-18827). The N-terminus of the NS2B protein was a serine protease, the C-terminus was a RNA helicase, and the peptide linker between them had 11 amino acid residues.

The present inventors found that the length and amino acid composition of the peptide linker, the presence or absence of glycosylation sites, the compatibility between the peptide linker and the two active molecules, and other factors, could affect the function and stability of the fusion proteins. The inventors believe that the peptide linker should have the following characteristics: 1) can make the linked proteins fold effectively into proper conformations, does not cause molecular dynamics change, and preferably comprises non-immunogenic natural amino acids. 2) should have the ability to prevent protease attack. 3) should try to avoid mutual impacts of the two fused proteins on each other.

In the absence of clear guidelines for the design of peptide linkers, the present inventors have developed new peptide linkers for the construction of fusion proteins, based on the inventors' long-term research experience, particularly those in studying Fc fusion proteins. Amazingly, the peptide linkers have advantage for maintaining the biological activity of proteins or polypeptides and have broad applicability and portability.

### DESCRIPTION OF THE INVENTION

The object of the present invention is to provide a novel peptide linker for constructing fusion proteins.

The first aspect of the present invention provides a peptide linker. The peptide linker comprises a flexible peptide and a rigid peptide. The flexible peptide consists of one or more flexible units, and the rigid peptide consists of one or more rigid units, wherein the flexible unit comprises two or more amino acid residues selected from Gly, Ser, Ala and Thr, and the rigid unit comprises the carboxyl terminal peptide (CTP) of human chorionic gonadotropin β subunit.

Preferably, the peptide linker is glycosylated. More preferably, the glycosylation site is located on CTP. Much more preferably, the glycosylation process is accomplished by expression in mammalian cells, such as Chinese hamster ovary cells, or other expression hosts having a suitable glycosylation modification system.

Further, the rigid peptide in the peptide linker of the present invention is located at the N-terminus or C-terminus of the flexible peptide. Preferably, the rigid peptide is located at the C-terminus of the flexible peptide. Specifically, the structural formula of the peptide linker of the present invention can be represented as F—R or R—F, wherein F and R represent the flexible peptide and the rigid peptide, respectively. Further, the flexible peptide preferably comprises 1, 2, 3, 4 or 5 flexible units, and the rigid peptide preferably comprises 1, 2, 3, 4 or 5 rigid units.

More preferably, the flexible unit comprises two or more G and S residues. Much more preferably, the amino acid sequence of the flexible unit has the general formula (GS)ₐ(GGS)_{b}(GGGS)_{c}(GGGGS)_{d} (SEQ ID NO: 37), wherein a, b, c and d represent the number of structural units composed of G and S residues, and are integers greater than or equal to 0, and a+b +c+d≥1.

Illustratively, each flexible unit is represented by Fᵢ and i=1, 2, 3, 4, 5, . . . n. In some embodiments of the invention, the flexible peptide preferably comprises, but is not limited to the following flexible units:

(i) F1:
(SEQ ID NO: 21)
GSGGGSGGGGSGGGGS;
(ii) F2:
(SEQ ID NO: 22)
GSGGGGSGGGGSGGGGSGGGGSGGGGS;
(iii) F3:
(SEQ ID NO: 23)
GSGGGGSGGGGSGGGGSGGGGSGGGGSGGGGSGGGGS;
(iv) F4:
(SEQ ID NO: 24)
GSGGGGSGGGGSGGGGS;
(v) F5:
(SEQ ID NO: 25)
GGGSGGGSGGGSGGGSGGGS;
(vi) F6:
(SEQ ID NO: 26)
GGSGGSGGSGGS.

The rigid unit is selected from the full length or fragment sequence of the C-terminal amino acids 113 to 145 of human chorionic gonadotropin β subunit. Specifically, the rigid unit comprises the amino acid sequence of SEQ ID no: 1 or its truncated form.

Preferably, CTP contains at least 2 glycosylation sites. For example, in a preferred embodiment of the present invention, CTP contains 2 glycosylation sites. Illustratively, CTP contains the N-terminal 10 amino acids of SEQ ID no: 1, i.e. SSSS*KAPPPS* (residues 6-15 of SEQ ID NO: 1), or CTP contains the C-terminal 14 amino acids of SEQ ID no: 1, i.e. S*RLPGPS*DTPILPQ (residues 20-33 of SEQ ID NO: 1). For another example, in another embodiment of the present invention, CTP contains 3 glycosylation sites. Illustratively, CTP contains the N-terminal 16 amino acids of SEQ ID no: 1, i.e. SSSS*KAPPPS*LPSPS*R (residues 6-21 of SEQ ID NO: 1). For other examples, in other embodiments of the present invention, CTP contains 4 glycosylation sites. Illustratively, CTP contains 28, 29, 30, 31, 32, or 33 amino acids, starting from position 113, 114, 115, 116, 117, or 118 and ending at position 145 of the human chorionic gonadotropin beta subunit. Specifically, CTP contains the N-terminal 28 amino acids of SEQ ID no: 1, i.e. SSSS*KAPPPS*LPSPS*RLPGPS*DTPILPQ (residues 6-33 of SEQ ID NO: 1). In this text, * represents a glycosylation site. Each possibility represents a separate embodiment of the present invention.

In other embodiments, the rigid units provided by the present invention have at least 70% amino acid sequence identity to native CTP. In other embodiments, the rigid units provided by the present invention have at least 80% amino acid sequence identity to native CTP. In other embodiments, the rigid units provided by the present invention have at least 90% amino acid sequence identity to native CTP. In other embodiments, the rigid units provided by the present invention have at least 95% amino acid sequence identity to native CTP.

Illustratively, each rigid unit is represented by Rᵢ and i=1, 2, 3, 4, 5, . . . , n. The rigid peptides described in some embodiments of the present invention may preferably comprise, but are not limited to, the following CTP rigid units.

(i) R1:
(SEQ ID NO: 27)
SSSSKAPPPSLPSPSRLPGPSDTPILPQ;
(ii) R2:
(SEQ ID NO: 28)
PRFQDSSSSKAPPPSLPSPSRLPGPSDTPILPQ;
(iii) R3:
(SEQ ID NO: 29)
SSSSKAPPPS;
(iv) R4:
(SEQ ID NO: 30)
SRLPGPSDTPILPQ;
(v) R5:
(SEQ ID NO: 31)
SSSSKAPPPSLPSPSR.

The rigid peptides of the present invention may further comprise two or three of the above CTP rigid units. In one embodiment of the present invention, the rigid peptide comprises two R3 rigid units: SSSSKAPPPSSSSSKAPPPS (SEQ ID NO: 32) (represented as R3R3). In another embodiment, the rigid peptide comprises three R4 rigid units: SRLPGPSDTPILPQSRLPGPSDTPILPQSRLPGPSDTPILPQ (SEQ ID NO: 33) (represented as R4R4R4).

In some preferred embodiments of the invention, the peptide linker has the amino acid sequence as shown in SEQ ID no: 2 (GSGGGGSGGGGSGGGGSGGGGSGGGGSSSSSKAPPPSLPSPSRLPGPSDTPILPQ) (represented as F2-R1).

In another preferred embodiment of the present invention, the peptide linker has the amino acid sequence as shown in SEQ ID no: 3 (GSGGGSGGGGSGGGGSPRFQDSSSSKAPPPSLPSPSRLPGPSDTPILPQ) (represented as F1-R2).

In another preferred embodiment of the present invention, the peptide linker has the amino acid sequence as shown in SEQ ID no: 4 (GSGGGGSGGGGSGGGGSSSSSKAPPPSLPSPSRLPGPSDTPILPQ) (represented as F4-R1).

In another preferred embodiment of the present invention, the peptide linker has the amino acid sequence as shown in SEQ ID no: 5 (GGGSGGGSGGGSGGGSGGGSSSSSKAPPPSLPSPSR) (represented as F5 -R5).

In another preferred embodiment of the present invention, the peptide linker has the amino acid sequence as shown in SEQ ID no: 6 (GSGGGGSGGGGSGGGGSSSSSKAPPPSSSSSKAPPPS) (represented as F4-R3R3).

In another preferred embodiment of the present invention, the peptide linker has the amino acid sequence as shown in SEQ ID no: 7 (GGSGGSGGSGGSSRLPGPSDTPILPQSRLPGPSDTPILPQSRLPGPSDTPILPQ) (represented as F6-R4R4R4).

Yet another aspect of the invention provides a fusion protein containing the peptide linker. The fusion protein comprises two biologically active molecules and a peptide linker linking the two active molecules. The structural formula of the fusion protein is expressed as K1-L-K2 or K2-L-K1, wherein K1 is the first biologically active molecule, L is the above-mentioned peptide linker, and K2 is the second biologically active molecule. The components of the fusion protein are sequentially linked from the N-terminus to C-terminus. Further, the active molecules may be selected from protein or protein domain, polypeptide, antibody or antibody fragment, preferably protein or protein domain, antibody or an antibody fragment.

Illustratively, the active molecule K1 of the fusion protein comprises protein or protein domain having biological function, polypeptide (e.g., especially soluble or membrane signal molecule), cytokine, growth factor, hormone, costimulatory molecule, enzyme, receptor, protein or polypeptide having ligand function for the receptor. The active molecule K2 is serum protein or protein domain that prolongs the circulation half-life, for example, human serum albumin (HSA), transferrin (TF), antibody/immunoglobulin Fc fragment, and so on.

Illustratively, the active molecule K1 of the fusion protein comprises toxin, enzyme, cytokine, membrane protein, or immunomodulatory cytokine. The active molecule K2 comprises antibody or antibody fragment. K1 and K2 are linked via the peptide linker to form an antibody fusion protein. For example, K2 is an antibody Fv fragment (VL or VH), and for another example, K2 is a single chain antibody (scFv).

Further, the biologically active molecule K1 comprises but is not limited to adenosine A1 receptor, angiotensin converting enzyme ACE, activin family, ADAM family, ALK family, a α-1-antitrypsin, programmed cell death associated protein family, nerve growth factor and receptor family, bone morphogenetic protein BMP and receptor family, complement factor, calcitonin, cancer associated antigen, cathepsin family, CCL chemokine and receptor family, CD superfamily, CFTR, CXCL chemokine and receptor family, EGF, epidermal growth factor EGF and receptor family, coagulation factor IIa, factor VII, factor VIII, factor IX, ferritin, fibroblast growth factor FGF and receptor family, follicle stimulating hormone, FZD family, HGF, glucagon, cardiac myosin, growth hormone, Ig, IgA receptor, IgE, insulin-like growth factor IGF and binding protein family, interleukin IL superfamily and its receptor superfamily, interferon INF family, iNOS, integrin family, kallikrein family, laminin, L-selectin, luteinizing hormone, MMP family, mucin family, cadherin superfamily, platelet-derived growth factor PDGF and receptor family, parathyroid hormone, serum albumin, T-cell-related receptor superfamily, TGF-α, transforming growth factor TGF-β superfamily, thyroid stimulating hormone, parathyroid stimulating hormone, tumor necrosis factor TNF superfamily and its receptor TNFRSF superfamily, urokinase, WNT signaling pathway family, thymosin α1, thymosin β4, VEGF, vascular endothelial growth factor VEGF and its receptor family.

In the preferred embodiments of the present invention, the active molecule K1 is human coagulation factor VII (FVII), human coagulation factor VIII (FVIII), GLP-1 analogue Exendin-4 (Ex4), human interleukin 7 (IL-7), or human growth hormone (hGH). The peptide linker L of the fusion protein is as shown in SEQ ID no: 2, 3, 4, 5, 6 or 7. The active molecule K2 is selected from the Fc fragments of human immunoglobulins IgG, IgM, and IgA. More preferably, the Fc fragments are derived from human IgG₁, IgG₂, IgG₃ or IgG₄. Furthermore, the Fc fragments may be wild-types or variants. The Fc variants contain at least one amino acid modification in the wild-type human immunoglobulin Fc domain. The variants have reduced effector functions (ADCC or CDC effect) and/or an enhanced binding affinity for the neonatal receptor FcRn. Further, in the preferred embodiments of the present invention, the Fc variants are preferably selected from the following groups: (i) vFcγ1: human IgG₁ hinge, CH2, and CH3 regions containing Leu234Val, Leu235Ala and Pro331Ser mutations (the amino acid sequence is as shown in SEQ ID no: 8); (ii) vFcγ2-1: human IgG₂ hinge, CH2, and CH3 regions containing Pro331Ser mutation (the amino acid sequence is as shown in SEQ ID no: 9); (iii) vFcγ2-2: human IgG₂ hinge, CH2, and CH3 regions containing Thr250Gln and Met428Leu mutations (the amino acid sequence is as shown in SEQ ID no: 10); (iv) vFcγ2-3: human IgG₂ hinge, CH2, and CH3 regions containing Pro331Ser, Thr250Gln and Met428Leu mutations (the amino acid sequence is as shown in SEQ ID no: 11); (v) vFcγ4: human IgG₄ hinge, CH2, and CH3 regions containing Ser228Pro and Leu235Ala mutations (the amino acid sequence is as shown in SEQ ID no: 12).

More preferably, in one embodiment of the present invention, the FVII-Fc fusion protein comprises, sequentially from the N- to C-terminus, FVII (having the amino acid sequence as shown in SEQ ID no: 13) , the peptide linker (having the amino acid sequence as shown in SEQ ID no: 2), and human IgG Fc (having the amino acid sequence as shown in SEQ ID no: 11).

More preferably, in one embodiment of the present invention, the FVIII-Fc fusion protein comprises, sequentially from the N- to C-terminus, FVIII (having the amino acid sequence as shown in SEQ ID no: 14), the peptide linker (having the amino acid sequence as shown in SEQ ID no: 2), and human IgG Fc (having the amino acid sequence as shown in SEQ ID no: 11).

More preferably, in one embodiment of the present invention, the Exendin-4-Fc fusion protein comprises, sequentially from the N- to C-terminus, Exendin-4 (having the amino acid sequence as shown in SEQ ID no: 15). the peptide linker (having the amino acid sequence as shown in SEQ ID no: 2, 3, 5 or 7), and human IgG Fc (having the amino acid sequence as shown in SEQ ID no: 11).

More preferably, in one embodiment of the invention, the IL-7-Fc fusion protein, sequentially from the N- to C-terminus, comprises IL-7 (having the amino acid sequence as shown in SEQ ID no: 16), the peptide linker (having the amino acid sequence as shown in SEQ ID no: 2), and human IgG Fc (having the amino acid sequence as shown in SEQ ID no: 11).

More preferably, in one embodiment of the present invention, the hGH-Fc fusion protein comprises, sequentially from the N- to C-terminus, hGH (having the amino acid sequence as shown in SEQ ID no: 17), the peptide linker (having the amino acid sequence as shown in SEQ ID no: 2), and human IgG Fc (having the amino acid sequence as shown in SEQ ID no: 11).

In other preferred embodiments of the invention, the active molecule K1 of the fusion protein is the antibody heavy chain variable region (VH), and K2 is the antibody light chain variable region (VL). K1 and K2 are linked by the peptide linker to form a single chain antibody (scFv).

In some preferred embodiments of the invention, the active molecule K1 of the fusion protein comprises a first antibody or antibody fragment, and the active molecule K2 comprises a second antibody or antibody fragment. K1 and K2 are linked by the peptide linker to form a bispecific antibody.

Preferably, in one embodiment of the present invention, K1 is a full-length double-chain anti-CD20 antibody, K2 is a single chain anti-CD3 antibody, and K1 and K2 are linked by the peptide linker to form a bispecific antibody. More preferably, the heavy chain of the anti-CD20 double-chain antibody contained in the bispecific antibody has an amino acid sequence as shown in SEQ ID no: 18 and the corresponding light chain has an amino acid sequence as shown in SEQ ID no: 19. The anti-CD3 single chain antibody contained in the bispecific antibody has an amino acid sequence as shown in SEQ ID no: 20. The peptide linker has an amino acid sequence as shown in SEQ ID no: 4 or 6. The heavy chain of the anti-CD20 double-chain antibody is linked to the anti-CD3 single chain antibody by the peptide linker.

Yet another aspect of the present invention also provides a method for preparing a fusion protein having the structural formula expressed as: K1 -L-K2 or K2-L-K1, wherein K1 is the first biologically active molecule, L is the peptide linker, and K2 is a second biologically active molecule. The components that make up the fusion protein are sequentially linked from the N- to the C-terminus. The active molecules may be selected from the groups including protein or protein domain, polypeptide, antibody or antibody fragment, preferably protein or protein domain, antibody or antibody fragment. The preparation method includes the step of allowing K1 and K2 to be linked by L. In the preferred embodiments of the present invention, the method includes the following steps:

(a) Ligate the DNA sequences encoding the first active molecule K1 and the second active molecule K2 through the DNA sequence of the peptide linker L to form a fusion gene.

(b) Introduce the fusion gene obtained in step (a) into a eukaryotic or prokaryotic expression host.

(c) Culture the high yield expression host screened and obtained in step (b) to express the fusion protein.

(d) Harvest the fermentation broth of step (c) and isolate and purify the fusion protein.

Illustratively, the peptide linkers are used to link the active proteins/polypeptides to serum proteins with long circulation half-lives, such as antibody/immunoglobulin Fc fragments, human serum albumin (HSA), transferrin (TF), etc. In one preferred embodiment of the present invention, the preparation method of the FVII-Fc fusion protein includes the step of linking the active molecule FVII and human IgG Fc by the peptide linker (SEQ ID no: 2). In one preferred embodiment of the present invention, the preparation method of the FVIII-Fc fusion protein includes the step of linking the active molecule FVIII and human IgG Fc by the peptide linker (SEQ ID no: 2). In another preferred embodiment of the present invention, the preparation method of the Exendin 4-Fc fusion protein includes the step of linking the active molecule Exendin-4 and human IgG Fc by the peptide linker (SEQ ID no: 2, 3, 5 or 7). In another preferred embodiment of the present invention, the preparation method of the IL-7-Fc fusion protein includes the step of linking the active molecule IL-7 and human IgG Fc by the peptide linker (SEQ ID no: 2). In another preferred embodiment of the present invention, the preparation method of the hGH-Fc fusion protein includes the step of linking the active molecule hGH and human IgG Fc by the peptide linker (SEQ ID no: 2).

Illustratively, the peptide linkers are used in the construction of bispecific antibodies. In the preferred embodiment of the present invention, the preparation method of the bispecific antibody of anti-CD3×CD20 includes the step of linking the double-chain anti-CD20 antibody and the single chain anti-CD3 antibody by the peptide linker (SEQ ID NO: 4 or 6).

In this present invention, technical novelities can be summarized as follows:

1. In the present invention, partof the peptide linker, CTP, contains multiple O-glycosyl side chains. It is capable of forming a relatively stable and rigid three-dimensional structure and thus more effectively separate the two partners of the fusion protein and eliminate the steric hindrance between them. In the construction of a series of fusion proteins composed of the active proteins and the Fc fragment, for example, the FVII-Fc fusion protein, the introduction of the rigid CTP unit into the peptide linker ensures that the N-terminally fused active protein does not affect the binding site of the Fc variant to FcRn, thus having no effect on the circulation half-life of the fusion protein. In addition, the protein A binding site of Fc is important for the purification step, and the introduction of the rigid CTP unit ensures that the N-terminally fused active protein will not “block” the protein A binding site. On the other hand, the introduction of the rigid CTP unit makes the 25 kD size of the Fc fragment not interfere with the folding of the N-terminally fused active protein, which prevents the decline or loss of the biological activity/function of the active protein. Many embodiments of the present invention indicate that the introduction of the rigid CTP unit makes the biological activity of the fusion proteins significantly improved. This can be explained as follows. The rigid CTP polypeptide possesses multiple glycosyl side chains. Compared with irregular coil of a flexible peptide linker such as (GGGGS)ₙ (SEQ ID NO: 38), CTP can form a stable three-dimensional conformation, which effectively increases the distance between the two fusion partners of the fusion protein. The spatial separation facilitates independent folding of the active protein and the Fc segment to form correct three-dimensional conformations, such that the active protein and the Fc segment would have no mutual impact on the biological activity of each other. This reduces the possibility of the decline or loss of the activity of the active protein due to misfolding and conformational alteration. Thus the biological activity of the fusion protein is increased.

2. The peptide linkers of the present invention have a wide range of applicability and portability. With a combination of rigid and flexible units, the peptide linkers are conferred with a conformation between completely rigid and fully flexible, and the specific rigidity or flexibility of the polypeptide varies depending on the ratio and arrangement order of the two sequences. As sequences are designed with different combinations of ratio and arrangement order of rigid and flexible units, the rigidity of the peptide linker can be finely regulated to meet different requirements in the construction of the fusion proteins.

3. CTP contains glycosylation sites. Highly sialylated and negatively charged CTP can resist the kidney to its clearance and further prolongs the half-life of the fusion protein. CTP can improve pharmacokinetic parameters, such as reducing the clearance rate, reducing the apparent distribution of volume, increasing AUC_{(0˜t)}, such that the bioavailability of the fusion protein increases. It is expected that the clinical dose will be reduced.

4. The glycosyl side chains of CTP has protective effect, which can reduce the sensitivity of the peptide linker to proteases. The fusion protein is not easy to be degraded in the linking region.

### Term Definitions

“Antibody fragment”: refers to an antigen-binding fragment of an antibody or antibody analogue, which typically comprises at least a portion of the antigen-binding region or variable region of the parental antibody, for example, one or more CDRs.

The “Fc” region: includes two heavy chain fragments, each of which comprises the CH2 and CH3 domains of an antibody. The two heavy chain fragments are held together by two or more interchain disulfide bonds and the inter-CH3 domain hydrophobic interactions.

The “Fv” region comprises the variable regions from both the heavy and light chains, but lacks constant regions.

“Single chain Fv antibody” (or scFv antibody): refers to an antibody comprising the VH and VL domains of an antibody, wherein these domains are present in a single polypeptide chain. In general, the Fv polypeptide additionally contains a polypeptide linker between the VH and VL domains that allows scFv to form the desired structure for antigen binding.

“Bispecific antibody”: refers to an antibody that comprises two Fv domains or scFv units such that the resulting antibody recognizes two different antigenic determinants.

“Antibody fusion protein”: refers to a product obtained by fusing an antibody fragment with another bioactive protein using genetic engineering techniques. Owing to many different fused proteins, the antibody fusion proteins have a variety of biological functions.

For example, an Fv-containing antibody fusion protein. As the Fab or Fv fragment is linked with certain toxins, enzymes, or cytokines, the biologically active molecules can be targeted to specific sites of the targeted cell, forming the so-called “biological missile.

For example, chimeric receptors. As the scFv antibody is fused with certain cell membrane protein molecules, the fusion proteins, known as chimeric receptors, can be expressed on the cell surface, giving the cells the capability to bind to a specific antigen.

For example, an Fc-containing antibody fusion protein. The antibody IgG Fc region is fused with the biologically active molecule to form an Fc fusion protein. The Fc fusion protein not only exerts the biological function of the active molecule but also inherits similar properties of the antibody, including prolonged plasma half-life and a series of effector functions specific to the Fc region. For example, on the one hand, the Fc region plays an important role in the eradication of pathogens. The Fc-mediated effector functions of IgG are carried out by two mechanisms: (1) After the Fc regions of IgG molecules bind to the cell surface Fc receptors (FcγRs), pathogens are broken down by phagocytosis or lysis or by killer cells through an antibody-dependent cell-mediated cytotoxicity (ADCC) pathway. (2) After the Fc regions of IgG molecules bind to C1q molecules of the first complement Cl complexes, the complement-dependent cytotoxicity (CDC) pathway is triggered, such that pathogens are lysed. On the other hand, the antibody Fc region bind to the FcRn receptor to prevent the antibody from entering into lysosome to be degraded. The fusion proteins containing the Fc region are endocytosed and protected by FcRn. These fusion proteins are not to be degraded, but again enter into the circulatory system, thereby increasing the in vivo half-lives of these fusion proteins. Moreover, FcRn shows activity in adult epithelial tissues, and it is expressed in epithelial cells of the intestine, pulmonary trachea, nasal cavity, vagina, colon, and rectum. The fusion proteins containing the Fc region can effectively shuttle the epithelial barrier through FcRn-mediated cell transduction.

“hCG-β carboxy terminal peptide (CTP)”: is a short peptide derived from the carboxyl terminus of human chorionic gonadotropin (hCG) beta subunit. Four kinds of reproduction-related polypeptide hormones, follicle stimulating hormone (FSH), luteinizing hormone (LH), thyroid stimulating hormone (TSH), and human chorionic gonadotropin (hCG) comprise the same alpha subunit and respective specific beta subunits. Compared with the other three hormones, hCG has a significantly prolonged half-life, which is mainly due to the specific carboxyl terminal peptide (CTP) on its β-subunit. CTP has 37 amino acid residues, which possesses four O-glycosylation sites terminating with a sialic acid residue. Highly sialylated, negatively charged CTP can resist to the clearance by the kidney, thereby prolonging the in vivo half-life of a protein (Fares F A et al., 1992, Proc Natl Acad Sci USA, 89: 4304-4308).

### BRIEF DESCRIPTION OF THE FIGURES

FIG. 1. Comparison of the bleeding times of HA mice administered with FP-A1 and NovoSeven® for 1 h and 2 h. Compared with the HA-N-1h group, *P<0.05, ***P<0.01; compared with the C57-NS group, #P<0.05, ###P<0.01.

FIG. 2. The active half-lives of FP-A1 and NovoSeven® in rats administered with warfarin.

FIG. 3. The curves of the RBG value changes in 0-216 h in db/db diabetic mice given single injection of FP-C1 and FP-C5.

FIG. 4. The effect of different doses of FP-C1 on HbA1c in db/db diabetes mice. Comparison of the FP-C1 group with the model group, *P<0.05, **P<0.01.

FIG. 5. RBG value changes of STZ-induced diabetes mice with single injection of FP-C1 during 0-240 h. Comparison of the FP-C1 group with the modle group, *P<0.05, **P<0.01; Comparison of the Duraglutide group with the modle group, ΔP<0.05, ΔP<0.01.

FIG. 6. The effect of FP-C1 on the body weight of mice fed with high fat diet.

FIG. 7. The effect of FP-C1 on glucose tolerance in mice fed with high fat diet (mean±SD, n=8). Compared with the normal group, #P<0.05, ##P<0.01; compared with the high fat group, *P<0.05, **P<0.01.

FIG. 8. The effect of FP-C1 on the serum insulin concentration of mice fed with high fat diet (means±SD, n=8). Compared with the normal group, #P<0.05, ##P<0.01; compared with the high fat group, *P<0.05, **P<0.01.

FIG. 9. The effect of FP-C1 on the insulin resistance index of mice fed with high fat diet (mean±SD, n=8). Compared with the normal group, #P<0.05, ##P<0.01; compared with the high fat group, *P<0.05, **P<0.01.

FIG. 10. The effect of FP-C1 on the cross-sectional area of fat cells in mice fed with high fat diet. A: normal group. B: high fat diet group. C: FP-C1 group.

FIG. 11. The ability of IL7-Fc fusion proteins FP-D1 and FP-D2 to stimulate mouse mononuclear cells to proliferate.

FIG. 12. The ability of hGH-Fc fusion proteins FP-E1 and FP-E2 to stimulate Nb2 cells to proliferate.

FIG. 13. The curves of blood drug concentration vs. time of hGH-Fc fusion proteins FP-E1 and FP-E2.

FIG. 14. The growth curves of all groups of rats administered with the hGH-Fc fusion protein FP-E1.

FIG. 15. The anti-CD3×CD20 bispecific antibody FP-F1 activated human PBMC cells to secret IFN-γ in a concentration-dependent manner.

FIG. 16. The anti-CD3×CD20 bispecific antibody FP-F1 activated human PBMC cells in a concentration-dependent manner.

FIG. 17. The efficacy of anti-CD3×CD20 bispecific antibody FP-F1 in killing subcutaneous transplantation tumor.

### EXAMPLES

### Example 1. Peptide Linkers Used to Construct Fusion Proteins

The present inventors constructed a series of fusion proteins K1-L-K2 containing peptide linkers. The composition of each fusion protein was shown in Table 1. The DNA sequences encoding the first active molecule K1 and the second active molecule K2 were ligated by the DNA sequence of the peptide linker L to constitute a fusion gene. Preferably, the codons of the DNA sequences were optimized for expression in CHO cells. Preferably, the sequences were generated by chemical synthesis. Two restriction sites, SpeI and EcoRI, were added at the 5′ and 3′ ends of the synthesized fragment, respectively, to facilitate insertion of the fusion gene obtained above into the specific site of an expression vector. After verified by sequencing, the fusion gene was digested with the corresponding restriction endonucleases and inserted into the corresponding cleavage sites of the expression plasmid PXY1A1, obtained by modifying PCDNA3.1 as a template. The expression plasmid containing the gene of the fusion protein was thus obtained. The PXY1A1 plasmid contained, but was not limited to, the following important expression elements: 1) human cytomegalovirus immediate early promoter and highly exogenous expression enhancer needed by mammalian cells; 2) double screening markers with kanamycin resistance in bacteria and G418 resistance in mammalian cells; 3) murine dihydrofolate reductase (DHFR) gene expression cassette. When the host cell type was DHFR gene deficient, methotrexate (MTX) could co-amplify the fusion gene and the DHFR gene (U.S. Pat. No. 4,399,216). The expression plasmid of the fusion protein was transfected into a mammalian host cell line. The preferred host cell line was DHFR enzyme-deficient CHO cells in order to achieve stable and high levels of expression (U.S. Pat. No. 4,818,679). After two days of transfection, the medium was replaced with a screening medium containing 0.6 mg/mL G418. Cells were seeded in the 96-well plate at a certain concentration (5000-10000 viable cells/well) for 10-14 days until large discrete cell clones appeared. The transfectants resistant to the selecting antibiotic were screened by the ELISA assay. Subclones with high level expression of the fusion protein were isolated by limiting dilution and use of the 96-well culture plate. For a fusion protein already validated as a good one by multiple means, it was appropriate to amplify the DHFR gene by MTX drug inhibition to achieve higher level of expression. In the growth medium containing increasing concentration of MTX, the transfected fusion protein gene was co-amplified with the DHFR gene. The highly expressed monoclonal cell strains obtained were cultured under fed-batch conditions in shake flasks or a 5-liter fermentor. The fusion protein was purified by protein A affinity chromatography and other ion exchange chromatography.

The inventors constructed a series of fusion proteins containing the present invention's peptide linkers with flexible and rigid units, and also constructed a variety of fusion proteins containing the peptide linkers with only flexible units of different lengths for comparison. For example, FVII-Fc fusion protein (FP-A1 containing CTP; FP-A2 and FP-A3 without CTP), FVIII-Fc fusion protein (FP-B1 containing CTP; FP-B2 and FP-B3 without CTP); Fc-fusion protein of Exendin-4 and its analogue (FP-C1, FP-C2, FP-C3, FP-C4 containing CTP; FP-C5 without CTP), IL7-Fc fusion protein (FP-D1 containing CTP; FP-D2 without CTP), hGH-Fc fusion protein (FP-E1 containing CTP; FP-E2 without CTP). In addition, the inventors also developed anti-CD2OXCD3 bispecific antibodies (FP-F1 and FP-F2 containing CTP). The composition of each fusion protein was shown in Table 1, and the amino acid sequence of each fusion protein was shown in the sequence listings section.

TABLE 1
The compositions of various fusion
proteins from N- to C-terminus

| Code for | | | |
|---|---|---|---|
| fusion | | | |
| protein | K1 | L | K2 |
| FP-A1 | FVII | F2-R1 | Fc (vFcγ₂₋₃) |
| FP-A2 | FVII | Fl | Fc (vFcγ₂₋₃) |
| FP-A3 | FVII | F3 | Fc (vFcγ₂₋₃) |
| FP-B1 | FVIII | F2-R1 | Fc (vFcγ₂₋₃) |
| FP-B2 | FVIII | F1 | Fc (vFcγ₂₋₃) |
| FP-B3 | FVIII | F3 | Fc (vFcγ₂₋₃) |
| FP-C1 | Exendin-4 | F2-R1 | Fc (vFcγ₂₋₃) |
| FP-C2 | Exendin-4 | F1-R2 | Fc (vFcγ₂₋₃) |
| FP-C3 | Exendin-4 | F5-R5 | Fc (vFcγ₂₋₃) |
| FP-C4 | Exendin-4 | F6-R4R4R4 | Fe (vFcγ₂₋₃) |
| FP-C5 | Exendin-4 | Fl | Fc (vFcγ₂₋₃) |
| FP-D1 | IL-7 | F2-R1 | Fc (vFcγ₂₋₃) |
| FP-D2 | IL-7 | F1 | Fc (vFcγ₂₋₃) |
| FP-E1 | hGH | F2-R1 | Fc (vFcγ₂₋₃) |
| FP-E2 | hGH | F1 | Fc (vFcγ₂₋₃) |
| FP-F1 | Anti-CD20 mAb | F4-R1 | Anti-CD3 ScFv |
| FP-F2 | Anti-CD20 mAb | F4-R3R3 | Anti-CD3 ScFv |

### Example 2. Preparation of Coagulation Factor FVII-Fc Fusion Proteins and Determination of Biological Activity and In Vivo Active Half-Life

### 2.1 Preparation and Identification of Coagulation Factor FVII-Fc Fusion Proteins

The stably expressing CHO cell strains of FP-A1, FP-A2 and FP-A3 obtained in Example 1 were cultured in shake flasks under fed-batch conditions for 10-14 days. The fusion proteins were purified by four steps of column chromatography: protein A affinity chromatography, multidimensional chromatography, anion exchange chromatography, and molecular sieve chromatography. The fusion proteins were then self-activated with incubation in a solution. The SDS-PAGE electrophoresis showed the following results. Under reducing conditions, the single-chained molecule of non-activated FP-A2 showed two obvious bands in the vicinity of 70-85 kDa and 40 kDa, indicating that protein degradation occurred and the fraction of the degraded fragments was about 20-30%. Under non-reducing conditions, the non-activated FP-A2 migrated to about 130 kDa together with another band of >200 kDa, indicating that some of the fusion proteins aggregated. Under reducing conditions, the single-chained molecule of non-activated FP-A3 was close to 100 kDa and there were no contaminant bands. Under non-reducing conditions, most of the non-activated FP-A3 proteins migrated to >200 kDa, indicating that FP-A3 was in the form of aggregates. Under reducing conditions, the single-chained molecule of non-activated FP-A1 was of 100-110 kDa and there were no obvious contaminant bands. Under non-reducing conditions, the non-activated FP-A1 migrated to 150 kDa. Under reducing conditions, the activated FP-A1 showed two clear bands, 74.3 kDa HC-L-CTP-Fc and about 24.0 kDa LC, respectively, and no other contaminant bands. Under non-reducing conditions, the activated FP-A1 migrated to 150 kDa, indicating that the fusion protein FP-A1 did not degrade significantly, did not form aggregates remarkably, and had higher thermodynamic stability and stronger ability of anti-proteolytic hydrolysis. This example demonstrated that the peptide linkers containing the rigid CTP unit increased the stability of the fusion proteins, which were less susceptible to degradation, and reduced the formation of aggregates.

### 2.2 Direct Determination of the Biological Activity of Fusion Proteins by the Clotting Assay

The determination of FVIIa biological activity by the clotting assay was achieved by correcting FVIIa-deficient plasma to prolong the clotting time. The kit (STAGO, Cat. No. 00743) was used for the assay. The assay was first to mix diluted human freeze-dried normal plasma of known FVII activity (Unicalibrator, Cat. No. 00625) with FVII-deficient plasma, measure the prothrombin time (PT), and establish a standard curve. The plasma for the FVII activity to be measured was diluted and then mixed with FVII-deficient plasma for PT measurement. The FVIIa activity of the test sample could be determined from the logarithmic equation between the percentage of activity C (%) and PT time t(s) fitted by the standard curve. The FVIIa activity was expressed as the percentage (%) of that of normal plasma. The corresponding relation between the percentage of activity of the standards (%) provided by the kit and the international unit (IU) of enzyme activity was 100%=1 IU, according to which the specific activity of FVII in the test sample could be calculated in units of IU/mg. The results showed that under optimum experimental conditions, the highest activities of FP-A1, FP-A2 and FP-A3 were about 20000 IU/mg, 4000 IU/mg and 7000 IU/mg, respectively. The experimental results showed that the type and length of the peptide linkers had a great effect on the activity and stability of FVII-Fc. The in vitro bioactivity of the fusion protein FP-A1 containing the peptide linker with the rigid unit was much higher than those of the fusion proteins FP-A2 and FP-A3 without rigid units. The results also showed that if only the length of the flexible peptide was extended, the activity of the fusion protein could not be improved effectively. The steric hindrance between the fused partners comprised the formation of the correct conformation of the fusion proteins, such that the stability of the fusion proteins decreased and the proteins were easy to form aggregates. This example demonstrated that the peptide linker containing a rigid CTP unit reduced the steric hindrance of the Fc domain and increased the activity and stability of the fusion protein.

### 2.3 Determination of In Vivo Bleeding Inhibition of FVII-Fc Fusion Proteins

The hemostatic effect of FVII-Fc fusion proteins was assessed by using hemophilia mice, which were the tail vein transection (TVT) bleeding model of homozygous hemophilia A mice with knockout of the FVIII factor gene (HA, Shanghai Research Center of the Southern Model Organisms). Male HA mice, 16-20 weeks old, were adaptively fed for one week and randomly divided into 3 groups, 6 mice per group. Two groups were given 300,000 IU/kg of FP-A1 and the other group was given 300,000 IU/kg of NovoSeven® (Novo Nordisk). Meantime, wild-type male C57BL/6J mice, 16-20 weeks old (Shanghai Research Center of the Southern Model Organisms), were used as the normal control group (n=6), and given an equal volume of physiological saline through tail vein injection. The two groups of HA mice given FP-A1 were subjected to tail-cutting tests at 1 h and 2 h after administration, respectively, and the HA mice given NovoSeven® were subjected to tail-cutting tests at 1 h after administration. The C57BL/6J normal control group (C57-NS group) was subjected to tail-cutting tests at 2 h after administration. All data were expressed as mean±standard error (±SEM). The t-test analysis was used to compare between the experimental groups. The analysis software was Graphpad Prism 5.0. P<0.05 was considered statistically significant.

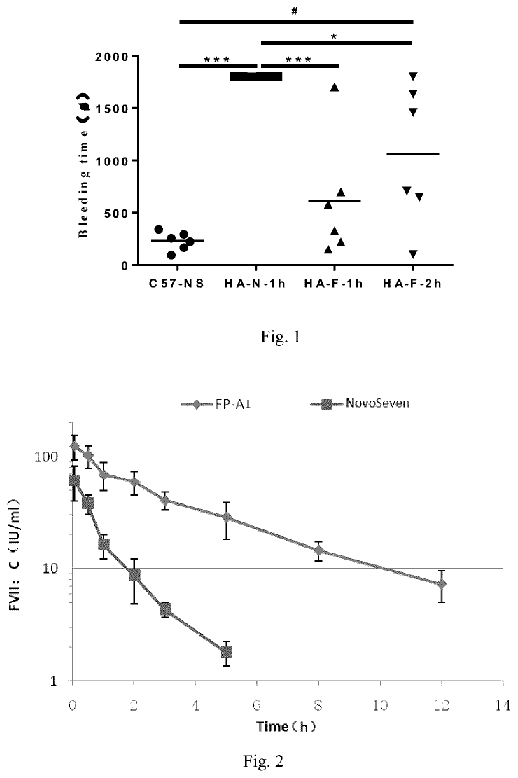

As shown in FIG. 1, after administration of NovoSeven® for 1 h, the bleeding time of the mice was 30 min, indicating that it had no hemostatic effect (HA-N-1h group). In contrast, after administration of FP-A1 for 1 h (HA-F-1h group) and 2 h (HA-F-2h group), FP-A1 was still effective in hemostasis, and the bleeding times were significantly shorter than that of the NovoSeven® group (P<0.05). This indicated that FP-A1 had a significantly longer active half-life compared with NovoSeven®.

### 2.4 Determination of In Vivo Active Half-Life of FVII-Fc Fusion Protein

In this study, we investigated the active half-life of FP-A1 in the warfarin-induced coagulation disorder rat model. According to the method reported in the literature (Joe Salas et al., 2015, Thrombosis Research, 135:970-976 or Gerhard Dickneite et al., 2007, Thrombosis Research, 119:643-651), SD rats (8-12 weeks age and 220 g body weight, Beijing Vital River Laboratory Animal Technology Co., Ltd.) were randomly divided into two groups, eight rats per group. After intragastric administration of warfarin (Orion Corporation, Finland, lot no. 1569755) at a dose of 2.5 mg/kg for 24 h, the rats were given intravenous administration of 10,000 IU/kg of FP-Al or NovoSeven® (Novo Nordisk), respectively. Blood was collected after administration for the FP-A1 group at 0.05 h, 0.5 h, 1 h, 2 h, 3 h, 5 h, 8 h, 12 h, respectively, and for the NovoSeven® group at 0.05 h, 0.5 h, 1 h, 2 h, 3 h, 5 h, respectively. With sodium citrate at a final concentration of 0.013 M as an anticoagulant, blood samples were centrifuged at 3000 rpm for 10 min to obtain the supernatant. The activity was determined by the method in section 2.2 and the active half-life was calculated.

As shown in FIG. 2, the active half-life of FP-A1 was calculated to be 3.03 ±0.35 h, and that of NovoSeven® was 1.01±0.16 h. Compared with NovoSeven® with equal activity, FP-A1 prolonged the active half-life in rats by about 3-fold. The plasma coagulation activity was about 40% at 3 h after single injection of FP-A1, while the coagulation activity of NovoSeven® decreased to 3% after 3 h. At 12 h after administration, the plasma coagulation activity of FP-A1 remained 7% or more.

The results in sections 2.3 and 2.4 showed that the fusion protein of the present invention, containing the peptide linker with the flexible and rigid CTP units, had a significantly prolonged active half-life, indicating that the peptide linker eliminated the blocking effect of the active protein FVII on the binding site of Fc for its receptor FcRn. The results also demonstrated that owning to the introduction of the peptide linker of the present invention, FVII formed the correct three-dimensional conformation, maintained high biological activity, and was not affected by the steric hindrance of the C-terminally fused Fc.

### Example 3. Production of Coagulation Factor FVIII-Fc Fusion Proteins and Determination of Biological Activity

### 3.1 Production and Identification of Coagulation Factor FVIII-Fc Fusion Proteins

The stably expressing CHO cell strains for FP-B1, FP-B2 and FP-B3 obtained in Example 1 were cultured for 7 to 12 days under fed-batch or semi-continuous culture conditions. The supernatant was harvested immediately for purification by protein A and/or VIII-select (GE) affinity chromatography. Under optimum culture conditions, the FP-B2 supernatant was passed through protein A and VIII-select (GE) two-step affinity chromatography columns, and the elute still contained multiple components. Under reducing conditions, the SDS-PAGE electrophoresis showed a main band of 180 kDa and multiple fragments of 40-100 kDa. Under non-reducing conditions, most of the purified proteins migrated to >300 kDa. This indicated that FP-B2 products were mostly in the form of aggregates, unstable and easy to be degradated. Under the same culture conditions, the supernatant of FP-B 1 was purified by protein A and VIII-select (GE) two-step affinity chromatography. Under reducing conditions, three clear bands appeared on the gel, which were single chain FVIII-Fc (190 kDa), light chain-Fc (105 kDa) and heavy chain (90 kDa), and there were no contaminant bands. Under non-reducing conditions, purified proteins FP-B1 and FP-B3 migrated to >200 kDa, while most of FP-B3 proteins stayed in the stacking gel, indicating that FP-B3 was also present in the form of aggregates.

It was reported that the lipid binding region of FVIII (2303-2332) was critical to its function, and that very small conformational changes in the region led to protein aggregation and loss of activity (Gilbert et al., 1993, Biochemistry, 32:9577-9585). The results of the present invention indicated that the peptide linker containing the rigid CTP unit could eliminate the steric hindrance of the C-terminal Fc on the FVIII lipid-binding region, such that the FVIII spatial conformation was almost unaffected. Thus, the protein aggregation was reduced, the protein stability was increased, and the bioactivity of the FVIII-Fc fusion protein was greatly improved.

### 3.2 Direct Determination of the Biological Activity of the FVIII-Fc Fusion Proteins by the Clotting Assay

The determination of the FVIII biological activity by the clotting assay was achieved by correcting FVIII-deficient plasma to prolong the clotting time. A factor VIII (FVIII) assay kit (the clotting method), STA®-Deficient FVIII (STAGO, Cat. No. 00725), was used. The method was first to determine the activated partial thromboplastin time (APTT) of normal human freeze-dried plasma (Unicalibrator, Cat No. 00625) of known coagulation factor VIII activity. The test instrument was a STAGO START® hemostasis analyzer. A standard curve was established. Then the FVIII-Fc fusion protein was mixed with the factor VIII-deficient plasma, and APTT values were determined. The logarithmic equation between the percentage of activity C (%) and APTT time t(s) fitted by the standard curve could be used to determine the activity of the test sample FVIII-Fc. The activity was expressed as the percentage (%) of that of normal plasma. The corresponding relation between the percentage of activity of the standards provided by the assay kit and the international unit (IU) of enzyme activity was 100%=1 IU, according to which the specific activity of FVIII in the test sample could be calculated, expressed as IU/mg. The results showed that under optimum experimental conditions, the highest activities of FP-B1, FP-B2 and FP-B3 were 10000 IU/mg, 150 IU/mg and 1300 IU/mg, respectively. Considering that most of the FP-B2 protein molecules were in the form of inactive aggregates or degraded fragments, the actual specific activities of FP-B2 and FP-B3 that were in active forms were not necessarily very different. This indicated that extending the length of the flexible peptide linker had a limited effect on improving the activity of the fusion protein FVIII-Fc. FP-B1 and FP-B2 showed a significant difference between the specific activities, which indicated that the rigid CTP unit in the peptide linker could reduce the steric hindrance of the Fc domain and improved the activity of the FVIII-Fc fusion protein.

### Example 4. Production of Exendin-4-Fc Fusion Proteins and Determination of Biological Activity and In Vivo Active Half-Life

### 4.1 In Vitro Biological Activity

The stably expressing CHO cell strains of FP-C1, FP-C2, FP-C3, FP-C4 and FP-C5 obtained in Example 1 were cultured in shake flasks under fed-batch conditions for 12-14 days. The fusion proteins were purified by Protein A affinity chromatography, and used for activity analysis. The purity of the fusion proteins was above 95%, and the molecular sizes were as expected. For the in vitro activity assay, referred to the literature (Zlokarnik G et al, 1998, Science, 279:84-88). The method was briefly described as follows. First, the human GLP-1R expression plasmid and the expression plasmid PGL-4.29 (Luc2P/CRE/Hygro) (Promega) carrying the CRE-Luc reporter gene were co-transfected into CHO-K1 cells. Then stable cell strains co-expressing both the plasmids were obtained after screening by antibiotic pressure. For the in vitro activity assay, 16000 cells per well in 200 μL medium were inoculated into the 96-well cell culture plate. The cells were cultured in DMEM medium containing 10% FBS for 16-24 h, until they grew to cover more than 90% of the bottom of the wells. The fusion proteins FP-C1, FP-C2, FP-C3, FP-C4 and FP-C5 were diluted with DMEM medium containing 10% FBS, and 10 μL was added to each well of the 96-well culture plate. The concentration gradients were set to 0.010, 0.020, 0.039, 0.078, 0.156, 0.313, 0.625, 1.25, and 2.5 nM. Meantime an equal concentration gradient of duraglutide (Eli Lilly, Cat. No. 9301897) was set as positive control. After the cells were incubated at 37° C., 5% CO₂ for 5-6 h, the supernatant was aspirated. The cells were washed slowly by adding 300 μL of PBS/well, then PBS was aspirated. 40 μL of lysis buffer was added and shaken for 15 min, and then 40 μL of luciferase substrate (Genomeditech (Shanghai) Co., Cat. No. GM-040501B, luciferase reporter gene detection kit) was added to each well. After 2 min reaction time, the fluorescence was measured at a wavelength of 560 nm using a multi-function microplate reader (SpectraMax M5 system, Molecular Device). A dose response curve was plotted based on the fluorescence values. The EC₅₀ value was calculated. The results were shown in Table 2. The EC₅₀ values of FP-C1, FP-C2, FP-C3, FP-C4 and FP-C5 were 0.03086 nM, 0.03156 nM, 0.03684 nM, 0.04012 nM and 0.03586 nM, respectively, and that of duraglutide was 0.02987 nM. The in vitro biological activities of CTP-containing FP-C1, FP-C2, FP-C3, FP-C4 and CTP-free FP-C5 were comparable. As the inventors understood, for a simple small molecule polypeptide such as Exendin-4, the steric hindrance of Fc to Exendin-4 was small, the effect of CTP that eliminated the steric hindrance and increased the activity of the fusion protein was not evident.

TABLE 2
Comparison of in vitro activity EC₅₀ values of fusion proteins

| Fusion | | | | | | |
|---|---|---|---|---|---|---|
| protein | FP-C1 | FP-C2 | FP-C3 | FP-C4 | FP-C5 | Duraglutide |
| EC₅₀ (nM) | 0.03086 | 0.03156 | 0.03684 | 0.04012 | 0.03586 | 0.02987 |

### 4.2 Blood Glucose Concentration Changes in Db/Db Diabetic Mice Given Single Injection of FP-C1 or FP-C5

Female diabetic db/db mice (Shanghai SLAC Laboratory Animals Co., Ltd.), 8 weeks old, weighing 42±2 g, were randomly divided into 3 groups, with 6 mice per each group. The drug-testing groups were injected subcutaneously with FP-C1 or FP-C5 at a dose of 3 mg/kg. The positive group was injected with 3 mg/kg of duraglutide (Eli Lilly, Cat. No. 9301897). The control group was injected with an equal volume of PBS buffer (10 mL/kg). Blood samples were collected from the tail vein at 0 h (before administration), 1 h, 2 h, 4 h, 6 h, 24 h, 48 h, 72 h, 96 h, 120 h, 144 h, 168 h, 192 h, 216 h (after administration). The blood glucose meter was used to measure random blood glucose (RBG) concentrations for recording the data. The blood glucose data were expressed as mean±standard deviation (mean±SD) and analyzed using the SPSS 18.0 statistical software package.

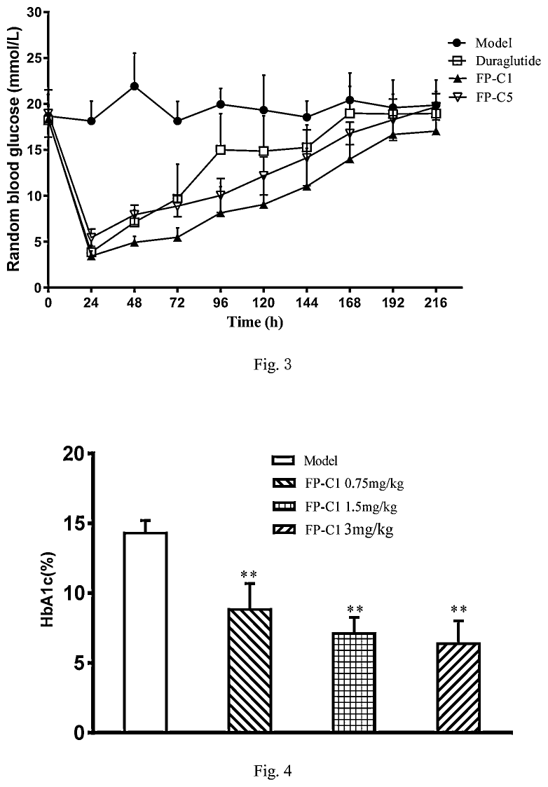

At the same dose, FP-C1, FP-C5 and the positive control drug duraglutide all had significant hypoglycemic effects. FIG. 3 showed the curves of 9-day mouse RBG values after administration. The hypoglycemic effect of duraglutide could only be maintained until day 4 (P>0.05). At 120 h after administration, the blood glucose level had no statistical difference compared with that of the control group. In contrast, FP-C1 could lower the blood glucose level of the mice for 168 h after administration, that was, the blood glucose level of the mice on the 7th day after administration was still statistically different from that of the control group (P<0.05). FP-C5 could maintain the hypoglycemic effect for 144 h after administration, that was, on the 6th day after administration the blood glucose level of the mice was still statistically different from that of the control group. Thus, compared with FP-C5, FP-C1 had a longer-term hypoglycemic effect in the diabetic mouse model. This indicated that the rigid CTP unit in the peptide linkers could further extend the in vivo functional half-life of Exendin-4.

### 4.3 Pharmacokinetic Characteristics of Exendin-4-Fc Fusion Proteins in Rats

Male SPF SD rats (Shanghai SIPPR-BK Laboratory Animal Co. Ltd.), 4 per group, were given a single subcutaneous injection of 0.5 mg/kg FP-C1 or FP-C5 after one week of pre-feeding. Blood was taken at 0 h (before administration), 2 h, 8 h, 24 h, 32 h, 48 h, 56 h, 72 h, 96 h, 120 h, 144 h (after administration), respectively, about 0.3 mL each time. The blood collection time points were marked as T₀, T₂, T₈, T₂₄, T₃₂ , T₄₈, T₅₆, T₇₂, T₉₆, T₁₂₀, and T₁₄₄, respectively. The blood was allowed to settle down. Then the serum was separated by centrifugation at 5000 rpm for 10 min, and the serum samples were stored at −70° C. and analyzed. The concentrations of fusion proteins were determined by the double-antibody sandwich ELISA assay. Used for coating was self-made or commercially available anti-Exendin-4 or GLP-1 N-terminal monoclonal antibody (e.g., Santa Cruz, Cat. No. SC-65389), and used as the detection antibody was self-made or commercially available horseradish peroxidase-labeled mouse anti-human-IgG-Fc monoclonal antibody (e.g., Sino Biological Inc., Cat. No. 10702-MM01E-50). The data were input into the analysis software PKSolver, and pharmacokinetic parameters such as T_{1/2}, Cmax and AUC_{(0˜t)} of the tested drug were obtained.

As shown in Table 3, the circulation half-life T_{1/2} of 0.5 mg/kg of FP-C5 in rats was 14.9±1.29 h, whereas the T_{1/2} of 0.5 mg/kg of FP-C1 in rats was 21.4±2.51 h. The maximum plasma concentration Cₘₐₓ of FP-C1 was significantly higher than that of FP-C5. In addition, by comparing AUC_{0˜t} (t=2 h, 5 h, 8 h, 24 h, 28 h, 32 h or 48 h) at different blood collection time points, we could see that at the same dose, the drug exposure of FP-C1 was significantly higher than that of FP-C5, that was, the absolute bioavailability of FP-C1 in rats was higher than that of CTP-free FP-C5. It was expected that the clinical dose of FP-C1 would also decrease.

TABLE 3
Pharmacokinetic parameters of single subcutaneous
injection of 0.5 mg/kg FP-C1 or FP-C5 in male SD rats

| | T_{1/2} (h) | Tₘₐₓ (h) | Cₘₐₓ (ng/mL) | AUC(0~∞) ng/mL · h |
|---|---|---|---|---|
| FP-C1 | 21.4 ± 2.51 | 24 | 700.908 | 35260.92 ± 2041.20 |
| FP-C5 | 14.9 ± 1.29 | 48 | 369.167 | 12535.06 ± 909.42 |

### 4.4 Random Blood Glucose and HbA1c Changes in Db/Db Diabetes Mouse After 10 Weeks of FP-C1 Treatment.

Female SPF-db/db mice (Shanghai slack experimental animal company), 8 weeks old, were fed for 1 week and divided into 4 groups with 6 in each group based on the RBG: model group, Low FP-C1 group (0.75 mg/kg), middle FP-C1 group (1.5 mg/kg), high FP-C1 group (3 mg/kg). Corresponding concentrations of the drug has been hypodermic injected, and PBS has been injected in the model group with 10 ml/kg. Mice in each group were treated with drug once a week for 10 weeks and subjected to blood glucose meter to measure the RBG in each time point. Data were recorded. Blood sampling points were 0 d (before drug treatment), 7 d, 14 d, 21 d, 28 d, 35 d, 42 d, 49 d, 56 d, 63 d and 70 d after drug treatment. At the 70^{th} day, mice in each group were blood sampled from the eyes after a 14 h fast and samples were subjected to the HbA1c kit and the corresponding H700 specific protein analyzer to detect the level of HbA1c. Results were shown as the percentage of HbA1c in the total blood proteins.

Date were presented as mean±SD which was generated by SP SS18.0. Differences between the means in the normal distribution were analysed by single factor variance. Homogeneity of variance was examined by Dunnett t-test and heterogeneity of variance was examined by Dunnett's T3 test. Abnormal distribution was examined by non-parametric test. P<0.05 showed significant statically difference.

From the variations of blood glucose of the mice in Table 4, mice in high, middle, low FP-C1 groups showed decreased levels of blood glucose compared with the model group, and their decreased blood glucose levels showed dose-dependent. These data indicated that FP-C1 effectively, continuously maintained the blood glucose level in db/db diabetes mice. Moreover, the hypoglycemic effect of the first administration and the last administration was similar, indicating that no drug resistance reaction occurred, which might lead to the anti-FP-C1 tolerance.

HbA1c was the product that glucose was bound to hemoglobin in the red blood cell, and showed proportional relationship with the glucose level in the blood. Because the red blood cell has a circulation half-life of 120 days, HbA1c represented the total glucose level in the blood in the 4-12 weeks before blood sampling, making up the shortcoming that the fasting blood sugar only reflected the transient blood sugar. So, HbA1c was the most critical indicator of monitoring blood glucose level and also one of the important factors to be considered in the clinical trial. The result of HbA1c in this example could reliably, stably reflect the blood glucose level 2-3 months before blood sampling. The amount of HbA1c in the mice with 10 weeks' drug treatment was shown in FIG. 4. The amount of HbA1c in the FP-C1 groups decreased significantly compared with that of the model group (P<0.01), showing dose-dependent. The amount of HbA1c in the high FP-C1 group decreased remarkably (6.38±1.63) but was still higher than that in the normal C57BL/6J mice (2.5%-3.5%), indicating that 3 mg/kg of FP-C1 would not lead to the occurence of long-term hypoglycemia. In conclusion, our results showed that FP-C1 chronically, effectively, stably controled the blood sugar level in the mice without increasing the risk of long-term hypoglycemia. It was consistent with the trend of blood glucose changes in Table 4.

TABLE 4
The effect of FP-C1 on random blood glucose in db/db mice

| | RBG (mmol/L) | | | | |
|---|---|---|---|---|---|
| Group | 7 d | 14 d | 21 d | 28 d | 35 d |
| Model | 19.5 ± 3.53 | 23.84 ± 3.28 | 21.9 ± 4.16 | 24.08 ± 2.18 | 21.92 ± 2.39 |
| Low-dose | 16.04 ± 1.98 | 18.5 ± 1.38** | 17.36 ± 3.87 | 19.58 ± 3.5* | 16.54 ± 3.41 |
| Middle-dose | 15.19 ± 2.65* | 17.34 ± 2.27* | 15.42 ± 3.6* | 17.82 ± 1.54** | 14.02 ± 3.42* |
| High-dose | 11.92 ± 1.95** | 15.26 ± 3.15** | 12.54 ± 3.97** | 15.36 ± 3.18** | 11.78 ± 4.2** |
| | RBG (mmol/L) | | | | |
| Group | 42 d | 49 d | 56 d | 63 d | 70 d |
| Model | 23.58 ± 3.51 | 23.66 ± 1.9 | 21.2 ± 4.72 | 23 ± 04.09 | 25.04 ± 5.24 |
| Low-dose | 16.02 ± 2.07** | 19.26 ± 2.17* | 13.92 ± 3.53* | 16.88 ± 3.92* | 17.46 ± 3.0* |
| Middle-dose | 14.3 ± 1.49** | 16.2 ± 2.86** | 12.6 ± 3.53** | 14.5 ± 4.37** | 15.14 ± 2.81** |
| High-dose | 12.12 ± 3.95** | 15.38 ± 2.43** | 9.46 ± 3.45** | 11.52 ± 3.93** | 11.76 ± 5.35** |

Note:
Compared with model group, *P < 0.05; **P < 0.01.

### 4.5 Pharmacodynamic Study of Single Injection of FP-C1 in STZ Induced Diabetes Mice

Male SPF mice (Shanghai slack experimental animal company) with the weight of 25±2 g were divided into the diabetes group and the control group based on their weights. Mice were fed for one week and subjected to a 18 h fast. The weights of the mice were recorded. The diabetes group was treated with 150 mg/kg 1% STZ, pH=4.4 by intraperitoneal injection. While the control group was treated the same volume of citric acid sodium citrate buffer. The RBG of the mice was recorded 10 days after the injection. The mice with RBG≥16.7 h were selected as the diabetes mice. 32 STZ-induced mice were selected and divided into 4 groups with 8 in each group to observe the hypoglycemic effect of test drugs on STZ-induced diabetic mice. 3 mg/kg FP-C1 and 3 mg/kg dulaglutide were injected subcutaneously, respectively. An equal volume (10 mL/kg) of PBS buffer was administered to the diabetes model group and the control group, respectively. The mice in each group are blood sampled from tail vein at 0 h (before drug treatment), 1 h, 2 h, 4 h, 6 h, 24 h, 48 h, 72 h, 96 h, 120 h, 144 h, 168 h, 192 h, 216 h (after drug treatment). Samples were subjected to the blood sugar meter to measure the RBG. Data were presented as mean±sd and analyzed by SPSS18.0. Differences between the means in the normal distribution were analysed by single factor variance. The homogeneity of variance was examined by LSD test and the heterogeneity of variance was examined by Dunnett's T3 test. Abnormal distribution was examined by non-parametric test. P<0.05 indicated significant statistical difference.

FIG. 5 showed the curves of the blood glucose concentration changes during 0-240 h in STZ-induced mice after single injection of FP-C1, FP-C1 decreased RBG effectively. The blood glucose level in the FP-C1 group decreased to the minimum at the 24 h after the FP-C1 treatment. Then it went back slowly but still showed significant difference compared with that of the model group (P<0.05).

### 4.6 Study on the Effect of FP-C1 on Weight Loss in Obese Mice Induced by High Fat Diet

### 1. Model Establishment and Drug Treatment

24 C57BL/6J male mice (Shanghai Slack Experimental Animal Company, SOCK(HU): 2012-0002) with 7 weeks old. Feeding environment: temperature 22-25° C., relative humidity 45-65%. Lighting time 12 h/d. The C57BL/6J mice were fed for one week and then divided into 3 groups based on the weights: NFD group, HFD group, FP-C1 group (HFD+FP-C1 0.3 mg/kg). The HFD group and the FP-C1 group were fed with high-fat diet (D12492 high fat diet, Research Dowts CO. USA). The NFD group was fed with normal fat diet. The FP-C1 group was injected with 0.3 mg/kg FP-C1 every 6 days, while the NFD group and the HFD group were injected with 10 ml/kg PBS buffer. The mice in each group had a 16 h fast after 96 days and the weights and blood sugar levels were recorded. Blood was sampled and subjected to 400 g centrifugation for 15 min to obtain the serum. After blood was taken, the mice were sacrificed by cervical dislocation, and the length from nose to anus (body length) was measured to calculate the Lee's index. The fat tissue around the epididymis was separated and weighed. The same tissue was stocked in 10% formaldehyde solution for further experiment.

### 2. Detection of Parameters

### 2.1 Body Weight and Obesity Degree

Mice were weighed every 6 days to draw the curve of body weight and the increased amount was recorded. Increased amount=body weight at the final weighing−body weight before grouping. Lee's index was used to describe the obesity degree.

### 2.2 Fat Mass and its Fraction

Analytical balance was used to weigh the fat tissue around the epididymis. Fat mass fraction was calculated as fat mass fraction=weight of fat tissue(mg)/fasting weight(g).

### 2.3 Oral Glucose Tolerant Test

After 84 days of experimentation, mice in each group were fasted for 16 hours (17:00 am-9:00 pm). The blood glucose meter was used to measure the fasting blood glucose (FBG). The body weights were recorded. 2 g/kg glucose solution was fed, and the blood sugar levels were detected after 30 min, 60 min, 90 min and 120 min of the gavage. The sugar tolerance curve was drawn and iAUC was calculated based on the modified curve.

### 2.4 Serum Yest

Automatic biochemical analyzer and corresponding kit were used to detect the concentration of TG and TC in the serum.

### 2.5 Parameters of Insulin and Islet Tolerance

ELISA was used to detect the concentration of insulin in the serum to calculate the insulin tolerance.

### 2.6 Pathological Examination of the Fat Tissue

Fat tissue around the epididymis was subjected to HE dyeing to visualize the fat tissue cell.

### 3. Statistics and Analysis

Data were presented as means±SD and analyzed by SPSS18.0. Differences between the means in the Normal distribution were analysed by single factor variance. Homogeneity of variance was examined by LSD test and heterogeneity of variance was examined by Dunnett's T3 test. Abnormal distribution was examined by non-parametric test. P<0.05 showed significant statistical difference.

### 4. Results

4.1 Effect of FP-C1 on Body Weight and Obesity Degree of Mice Fed with High-Fat Diet

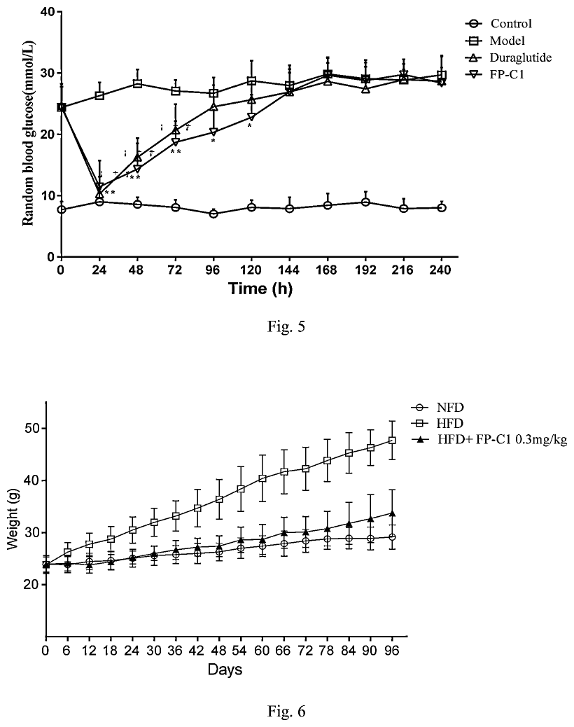

Compared with those in the NFD group, the body weight, the increased body weight and Lee's index all significantly increased (P<0.01). FP-C1 effectively decreased the body weight, the increased body weight, and Lee's index (P<0.01). Data were shown in Table 5 and FIG. 6.

TABLE 5
The effect of FP-C1 on body weight and
Lee's index in high fat diet induced obese mice

| | Final body | Body weight | |
|---|---|---|---|
| Group | weight (g) | gain (g) | Lee's index |
| NFD | 29.14 ± 2.31 | 5.24 ± 1.09 | 322.7 ± 11.03 |
| HFD | 47.7 ± 3.74^{##} | 23.9 ± 2.57^{##} | 348.78 ± 10.21^{##} |
| FP-C1 | 33.74 ± 4.5** | 9.81 ± 4.24** | 327.67 ± 10.28** |

Note:
Compared with normal group, ^{#}P < 0.05, ^{##}P < 0.01;
Compared with HFD group, *P < 0.05, **P < 0.01

### 4.2 Effect of FP-C1 on the Fat Mass and Mass Fraction Around the Epididymis

As shown in Table 6, compared with the NFD group, the mice in HFD group showed more fat mass and mass fraction around the epididymis (P<0.01). Compared with the HFD group, mice in FP-C1 group showed decreased fat mass and mass fraction around the epididymis (P<0.05).

TABLE 6
The effect of FP-C1 on
Epididymal fat and its index

| | Epididymal | Epididymal fat |
|---|---|---|
| Group | fat (g) | index (mg/g) |
| NFD | 0.68 ± 0.12 | 25.29 ± 3.83 |
| HFD | 2.91 ± 0.45^{##} | 64.1 ± 10.85^{##} |
| FP-C1 | 1.54 ± 0.69* | 46.09 ± 14.77* |

Note:
Compared with normal group, ^{#}P < 0.05, ^{##}P < 0.01;
Compared with HFD group, *P < 0.05, **P < 0.01

### 4.3 Effect of FP-C1 on the Concentrations of TG and TC in the Serum

Compared with NFD group, the concentrations of TG and TC in the serum in the HFD group increased significantly (P<0.01). Compared with HFD group, the concentrations of TG and TC in the serum in the FP-C1 group decreased significantly (P<0.01). Data shows in Table 7.

TABLE 7
The effect of FP-C1 on serum TG and
TC in high fat diet induced obese mice

| Group | TG (mmol · L⁻¹) | TC (mmol · L⁻¹) |
|---|---|---|
| NFD | 1.81 ± 0.31 | 4.34 ± 0.25 |
| HFD | 2.93 ± 0.33^{##} | 7.77 ± 0.85^{##} |
| FP-C1 | 1.98 ± 0.38** | 5.78 ± 0.85** |

Note:
Compared with normal group, ^{#}P < 0.05, ^{##}P < 0.01;
Compared with HFD group, *P < 0.05, **P < 0.01

4.4 Effect of FP-C1 on Glucose Tolerance in Mice Fed with High Fat Diet

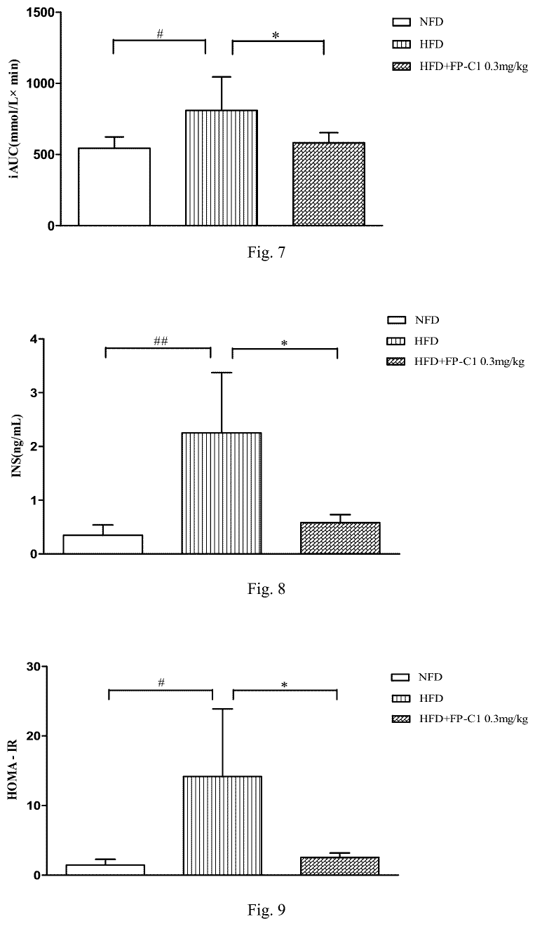

As shown in FIG. 7, iAUC in the HFD group is significantly higher than that in NFD group (P<0.05). Compared with the HFD group, iAUC in the FP-C1 group decreases significantly (P<0.05).

4.5 Effect of FP-C1 on Serum Insulin Concentration and Insulin resistance Index in Mice Fed with High Fat Diet

Compared with those in the NFD group, the insulin concentration and the insulin resistance index in the HFD group increased significantly, which meant that the mice obviously have already obtained insulin tolerance, and they would secreted more insulin to to produce hyperinsulinemia. Compared with those in the HFD group, FP-C1 decreased the INS concentration (P<0.05) and improved the HOMA-IR (P<0.05) significantly. Results were shown in FIG. 8 and FIG. 9.

### 4.6 Pathomorphological Examination

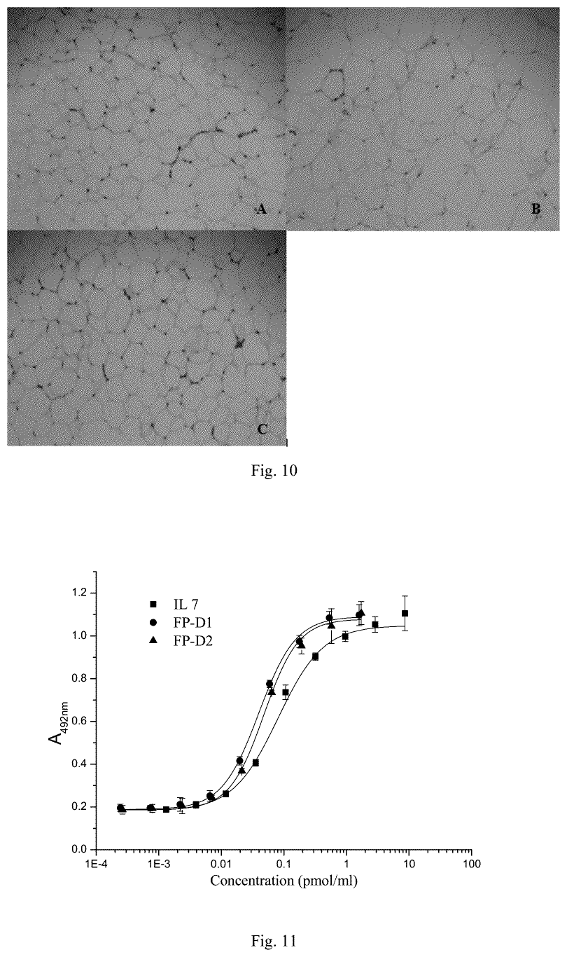

HE dyeing showed that the cross-sectional area of fat cells around the epididymis in the HFD group increased significantly compared with the NFD group. Compared with that in the HFD group, the cross-sectional area of fat cells around the epididymis in the FP-C1 group decreased significantly. Results were shown in FIG. 10.

In conclusion, FP-C1 could control the body weight of the HFD induced fat mice and has the anti-obesity effect.

### Example 5. Production of IL-7-Fc Fusion Proteins and Determination of Biological Activity and In Vivo Active Half-Life

### 5.1 In Vitro Biological Activity

The stably expressing CHO cell strains of FP-D1 and FP-D2 obtained in Example 1 were cultured in shake flasks under fed batch conditions for 12-14 days. The fusion proteins were purified by Protein A affinity chromatography. The purity of both the fusion proteins was above 95%, and the molecular sizes were also as expected. The fusion proteins were then used for activity analysis. The in vitro biological activity analysis of IL-7 and its fusion proteins was performed as follows. The mouse spleen-derived mononuclear cells were activated by concanavalin A (ConA), and then 100 μL of the cells were seeded into each well of a 96-well plate, followed by addition of a series of concentration gradient of FP-D1 or FP-D2. The cells were cultured at 37° C., 5% CO₂ for 72 h, and after addition of 20 μL of MTT reagent, were continued to culture for 4 h. The medium was aspirated and 100 μL of dimethyl sulfoxide (DMSO) was added to each well. The absorbance at 492 nm was used to determine the proliferation of the cells. Three replicates were performed for each concentration gradient, and each replicate was measured twice. Recombinant IL-7 (hIL-7, Sino Biological Inc.) was used as positive control and the medium was used as negative control. FIG. 11 showed the ability of hIL-7, FP-D1 and FP-D2 fusion proteins to stimulate the proliferation of mouse mononuclear cells. Based on the dose response curves plotted, the half maximal effective concentration values (EC₅o) of the FP-D1 and FP-D2 fusion proteins were 0.039 and 0.048 nM, respectively. The recombinant fusion proteins exhibited better in vitro bioactivity than hIL-7 that E. coli expressed (EC₅₀ value of 0.08 nM in terms of molarity).

### 5.2 Determination of Pharmacokinetic Parameters of IL-7-Fc Fusion Proteins

Male SPF SD rats (Shanghai SIPPR-BK Laboratory Animal Co. Ltd.), 3 per group, were given a single intravenous (iv) or subcutaneous (sc) injection of 2 mg/kg FP-D1 or FP-D2 after one week of pre-feeding. The changes of blood drug concentration with time were investigated. At 0 h, 3 h, 6 h, 24 h, 48 h, 72 h, 96 h, 120 h, 126 h, 144 h, and 172 h after injection, blood was collected, about 0.3 mL each time. The blood was allowed to settle down. Then the serum was separated by centrifugation at 5000 rpm for 10 min and taken for assay. The fusion protein concentration at each time point was determined by an ELISA method specific for human IL-7. The data were analyzed by the PKSolver software, and pharmacokinetic parameters such as T_{1/2} and AUC_{(0˜t)} were calculated. The results were shown in Table 8.

TABLE 8
Pharmacokinetic parameters of fusion proteins

| | | | AUC 0-inf_obs | | Vz/F_obs | | Cl/F_obs | |
|---|---|---|---|---|---|---|---|---|
| | T_{1/2}(h) | | (ng/mL * h) | | ((μg)/(ng/mL)) | | ((μg)/(ng/mL)/h) | |
| Drug | iv | sc | iv | sc | iv | sc | iv | sc |
| FP-D1 | 18.760 ± | 19.870 ± | 24458.54 ± | 10310.28 ± | 0.526 ± | 1.293 ± | 0.020 ± 0.006 | 0.045 ± 0.005 |
| | 1.109 | 2.287 | 8739.60 | 1364.23 | 0.143 | 0.173 | | |
| FP-D2 | 12.86 ± | 13.81 ± | 12025.16 ± | 4497.43 ± | 0.79 ± | 0.11 ± | 0.04 ± 0.01 | 2.20 ± 0.75 |
| | 4.32 | 4.47 | 2074.52 | 571.93 | 0.32 | 0.01 | | |

The data showed that the elimination half-lives of FP-D2 were 12.86 h (iv) and 13.81 h (sc), respectively, which fit with linear metabolic kinetics. The elimination half-lives of FP-D1 were 18 h (iv) and 19 h (sc), respectively, slightly longer than those of FP-D2. The overall clearance rate (Cl) of FP-D1 by intravenous administration was less than that of FP-D2, indicating that after FP-D2 was modified to generate FP-D2, the in vivo clearance rate of FP-D1 was slowed down compared with that of FP-D2. In addition, we also found that due to the introduction of the CTP structure, the apparent volume of distribution of FP-D1 was reduced. This indicated that FP-D1 was less distributed in tissues and had a higher concentration in the blood, thus having a higher in vivo exposure. FP-D1 had the rigid CTP structure compared with FP-D2, which reduced the clearance, reduced the apparent volume of distribution, and increased AUC_{(0˜t)}. Therefore, the bioavailability of FP-D1 was higher, and the efficacy of FP-D1 might be better than that of FP-D2. It was expected that the clinical dose of FP-D1 would also decrease. Thus, FP-D1 exhibited excellent performance in terms of biological activity and pharmacokinetics. The results of this experiment indicated that the highly sialylated, negatively charged CTP in the peptide linkers could resist the clearance of the kidney, further prolong the half-lives of the fusion proteins, and increase the bioavailability of the fusion proteins.

### Example 6. Production of hGH-Fc Fusion Proteins and Determination of Biological Activity and In Vivo Half-Life

### 6.1 Determination of In Vitro Biological Activity of Fusion Proteins by MTT Assay

The stably expressing CHO cell strains of FP-E1 and FP-E2 obtained in Example 1 were cultured in shake flasks under fed-batch conditions for 12-14 days. The fusion proteins were purified by Protein A affinity chromatography. The purity of the fusion proteins was above 95%, and the molecular sizes were as expected. The fusion proteins were then used for activity analysis. In vitro hGH biological activity could be determined by measuring proliferation of Nb2 rat lymphoma cells. Since the Nb2 cells responded to the stimulation of hGH on the lactating receptors of the Nb2 cells to proliferate, the Nb2 cell proliferation assay could be used to evaluate the biological activity of growth hormone (Uchida H et al., 1999, J Mol Endocrinol, 23: 347-353).

Nb2-11 rat lymphoma cells (ATCC) were cultured in Fischer's medium containing 10% FBS. The fusion protein was diluted with serum-free medium, diluted from 1000 ng/mL to 8 gradients at 1:3 ratio, and added to a 96-well plate, 100 μL per well, with the last column using the medium as negative control. The cells growing in the log phase were washed with serum-free medium, and adjusted to a density of 3×10⁶ cells/mL. 100 μL cell suspension was added to each well of the above 96-well plate. The cells were continued to culture for 48 h in a 37° C., 5% CO₂ incubator, and cell proliferation was measured using a CCK-8 kit (Cell Counting Kit, Cat. No. 40203ES80, Shanghai Yisheng Biotechnology Co., Ltd.). The absorbance at 450 nm was measured using a microplate reader and the OD readings were plotted against the concentrations of the fusion protein. The biological activity of the fusion protein could be determined from the resulting dose response curve.

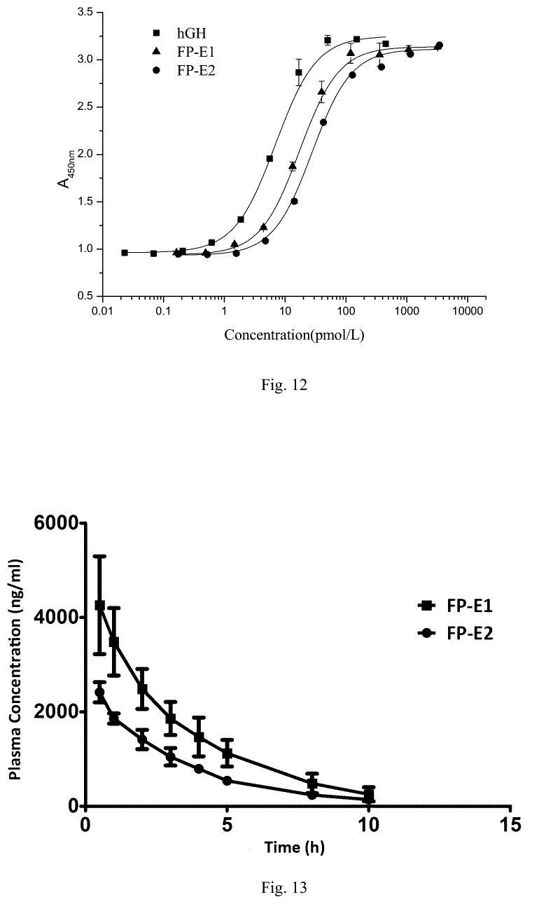

FIG. 12 showed the ability of hGH fusion proteins to stimulate the proliferation of Nb2 cells. Table 9 showed the EC₅₀ values for different fusion proteins. Since the amino acids at the C-terminus of growth hormone were closely related to its function, direct linking of Fc to the C-terminus of hGH might affect its biological activity. As the peptide linker was added between hGH and Fc, the activity of the hGH fusion protein was increased. As can be seen from the results, the activity of FP-E1 was nearly doubled compared with that of FP-E2. This might be due to the fact that CTP peptide did not only play its role in prolonging the half-life of the fusion protein, but alsothe rigid structure of CTP peptidetogether with a flexible peptide acted as a linker connecting Fc to the target protein. This novel peptide linker facilitated folding of the fusion protein into a stable three-dimensional structure, thus increasing the biological activity of hGH.

TABLE 9
EC₅₀ values of hGH fusion proteins

| Fusion protein | hGH | FP-E1 | FP-E2 |
|---|---|---|---|
| EC₅₀ (pM) | 6.94 | 17.3 | 27.84 |

### 6.2 In Vivo Circulation Half-Life

Male SPF SD rats (Shanghai SIPPR-BK Laboratory Animal Co. Ltd.), weighing about 290 g, were divided into groups after one week of pre-feeding, with 3 in each group, and were given a single intravenous injection of 0.176 mg/kg FP-E1 or FP-E2. The changes of blood drug concentration with time were investigated. For the control group and the drug-administered group, blood was collected at 0 h, 0.5 h, 1 h, 2 h, 3 h, 4 h, 5 h, 8 h, 10 h, 24 h, 48 h, and 72 h after administration. The blood was placed at room temperature for 30 min and then centrifuged at 5000 rpm for 10 min. The serum was separated and stored at -20° C. Serum hGH levels at various time points were determined using an ELISA assay specific for hGH. The main pharmacokinetic parameters of each group were calculated by the PKSolver software. The pharmacokinetic parameters of each group were shown in Table 10.

TABLE 10
Pharmacokinetic parameters of hGH fusion proteins

| | | Tmax | | | MRT_{(0-∞)} | | Cl(μg)/(ng/mL)/ |
|---|---|---|---|---|---|---|---|
| Drug | T_{1/2}(h) | (h) | Cmax (ng/mL) | AUC₍₀₋ₜ₎ | (h) | Vz(μg)/(ng/mL) | h |
| FP-E1 | 2.39 ± 0.37 | 0.5 h | 4259.4396 | 16483.295 ± 4483.39 | 3.34 ± 0.43 | 0.027 ± 0.002 | 0.008 ± 0.002 |
| FP-E2 | 2.57 ± 0.04 | 0.5 h | 2414.7499 | 8987.14 ± 986.33 | 3.33 ± 0.01 | 0.054 ± 0.008 | 0.015 ± 0.002 |

From the results, the in vivo half-lives of FP-E1 and FP-E2 fusion proteins were 2.6 h and 2.4 h, respectively. The half-lives were basically the same, and this could also be seen from the MRT parameters. The times that FP-E1 and FP-E2 stayed in the body were also close. According to the blood drug concentration-time curve of FIG. 13, the blood concentration of FP-E1 was always higher than that of FP-E2. It was speculated that structural changes of FP-E1 resulted in the changes in the properties of its pharmacokinetics. The total body clearance of FP-E1 was only half of that of FP-E2, indicating that the in vivo clearance of FP-E1 was relatively slow. The apparent volume of distribution of FP-E2 was twice that of FP-E1, indicating that FP-E2 was rapidly distributed into tissues after entering the body, causing its blood concentration to be lower than that of FP-E1. FP-E1 had the rigid CTP structure compared with FP-E2, which reduced the clearance, increased AUC_{(0˜t)}, and reduced the apparent volume of distribution. This indicated that FP-E1 was less distributed in tissues and had higher concentration in the blood, resulting in higher in vivo exposure. So the structural advantage of FP-E1 was reflected not only in the superior pharmacokinetic parameters, but also in that its efficacy could be better than that of FP-E2. FP-E1 had a higher blood concentration due to the structural modifications, which meant that its bioavailability was higher. So FP-E1 was better than FP-E2, and its clinical dose could be expected to decrease. Thus, FP-E1 exhibited superior performance in terms of biological activity and pharmacokinetics.

### 6.3 Determination of In Vivo Biological Activity of Fusion Proteins

Male Sprague-Dawley rats (SPF grade with four weeks age and 60-80 g body weight) were provided by the Experimental Animal Center of China National Institutes for Food and Drug Control.

The pituitary gland was removed by surgery with two week's recovery period. Before administration, the qualified healthy animals were selected, whose body weight changes were less than ±10% of the preoperative weights. The pituitary gland-removed rats were randomly divided into 6 groups, 5 animals per group. Regular rhGH (trade name norditropin, Novo Nordisk A/S, activity of 3 IU/mg) was used as the positive reference drug 1 (Y1) in this experiment. PEG-rhGH (Changchun GeneScience Pharmaceuticals Co., Ltd., activity of 6 IU/mg) was used as the positive reference drug 2 (Y2). Low, median and high doses of FP-E1 were set at 5 mg/kg/14 d, 15 mg/kg/14 d, and 45 mg/kg/14 d, respectively. Based on the molecular weight and the number of molecules, the median dose of FP-E1, 15 mg/kg/14 d, was comparable to the positive reference drugs Y1 and Y2. The mode of administration was subcutaneous injection in the neck. Different doses of FP-E1 fusion protein and Y2 were administered to each rat once on the first day and the other on the eighth day. Y1 was administered once a day for 14 consecutive days.

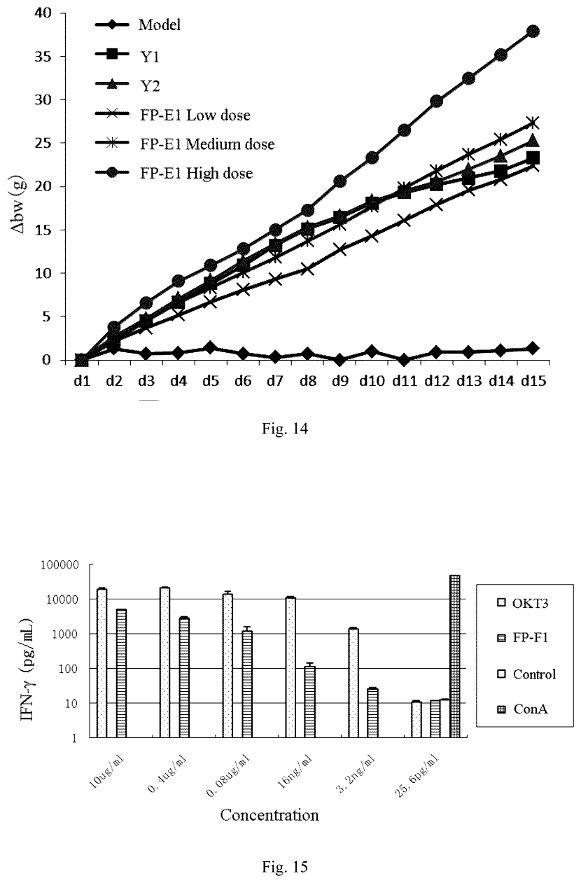

Each rat was weighed daily after administration. All rats were sacrificed by carbon dioxide asphyxiation on the 15th day, and weighed. The difference between the day i (di) body weight (bwᵢ) of each animal and the body weight before administration, i.e. the day 1 (d1) body weight (bw1), was the increased body weight. If necessary, an autopsy could be performed at the end of the experiment. The sella region was cut, and the presence or absence of pituitary residue was examined by the naked eye. The animals with residual pituitary gland were removed. The weight gain calculation formula was: Δbw=bwᵢ−bw₁, wherein Δbw was the weight gain; bw₁ was the d1 body weight before administration; and bwᵢ was the day i body weight after administration. The measurement data were expressed as mean±standard deviation (M±SD), and the results were shown in Table 11. FIG. 14 showed the growth curves of all groups after administration.

TABLE 11
Body weight changes of rats before and after hGH
fusion protein administration (M ± SD, n = 7)

| | Total dose | bw (g) before | Δbw (g) after | |
|---|---|---|---|---|
| | for 2 weeks | administration | administration | |
| Group | mg/kg | d1 | d8 | d15 |
| Control | 0 | 72.4 ± 6.9 | 0.7 ± 1.4 | 1.3 ± 1.7 |
| Y1 | 5.8 | 73.1 ± 5.2 | 15.1 ± 2.5 | 23.3 ± 4.4 |
| Y2 | 2.9 | 72.2 ± 6.4 | 15.3 ± 2.3 | 25.3 ± 4.3 |
| Low dose | 5 | 74.3 ± 6.8 | 10.5 ± 1.3 | 22.4 ± 2.9 |
| of FP-E1 | | | | |
| Median dose | 15 | 72.4 ± 4.8 | 13.7 ± 3.1 | 27.3 ± 6.8 |
| of FP-E1 | | | | |
| High dose | 45 | 73.6 ± 5.9 | 17.3 ± 2.1 | 37.9 ± 3.0 |
| of FP-E1 | | | | |

As could be seen from the results, compared with the control group, each of the administration groups had its body weight significantly increased on the 8th day after administration, but the differences of Δbw between the administration groups were not significant. On the 15th day after administration, FP-E2 had a very significant promotive effect on the body weight gain of rats. The weight gain (Δbw) of pituitary-removed rats induced by the high dose FP-E1 was about 1.5 times those of the rhGH (Y1) and PEG-rhGH (Y2) groups. The weight gain induced by the median dose FP-E1 was slightly larger than that of the Y1 group, and was basically equal to that of the Y2 group. We found that the high dose FP-E1 group had their body weights significantly increased after the second administration, and FP-E1 showed a more significant promotive effect on weight gain than PEG-rhGH. However, within one week after the first administration, the Abw values of the high dose FP-E1 and PEG-rhGH groups were not statistically different. Based on the analysis of the Abw differences, it was deduced that FP-E1 should have a longer in vivo active half-life than PEG-rhGH. Thus, after repeated administration, this in vivo drug accumulation effect caused the upward trend of the growth curve of the FP-E1 group steeper after the second administration.

### Example 7. Production and Biological Activity Analysis of anti-CD3×CD20 Bispecific Antibodies

### 7.1 Preparation and Identification of Anti-CD3×CD20 Bispecific Antibodies

The stably expressing CHO cell strains of FP-Fl and FP-F2 obtained in Example 1 were cultured in shake flasks under fed-batch conditions for 10-14 days. The bispecific antibodies were purified by Protein A affinity chromatography. The purity of both the fusion proteins was more than 95%, and the molecular sizes were as expected. The proteins were then used for activity analysis.

### 7.2 Determination of In Vitro Activity of Anti-CD3×CD20 Bispecific Antibodies

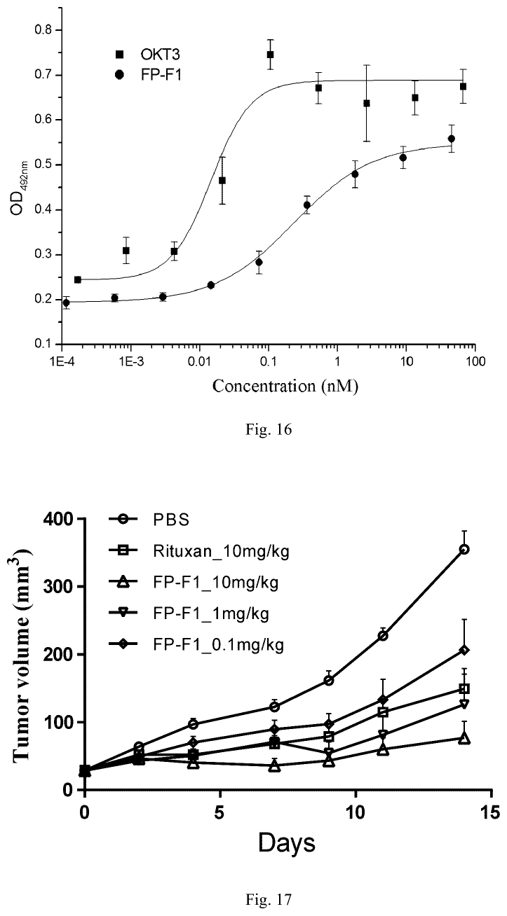

PBMCs were prepared from fresh human blood by density gradient centrifugation and resuspended at 5×10⁶ cells/mL with medium containing 10% heat inactivated FBS for later use. The antibody (anti-CD3 monoclonal antibody OKT3 or bispecific antibody FP-F1) was diluted to 2μg/mL using complete medium, and then diluted to 8 gradients at 1:5 ratio. The diluted antibody was added to a 96-well plate at 100 μL per well in triplicate. The medium was used as negative control and ConA was used as positive control. The PBMC cell suspension prepared as above was added to the 96-well plate at 100 μL/well, and cultured at 37° C., 5% CO₂ for 72 h. After incubation, the culture supernatant in the 96-well plate, 100 μL per well, was carefully aspirated, and the IFN-γ content in the supernatant was measured by an IFN-γ ELISA kit (BD Biosciences) (FIG. 15). 10 μL of the CCK-8 reagent was added to each well of the 96-well plate, and the incubation was continued for 4 h. Using the antibody concentration as the X-axis and the absorbance value at 492 nm as the Y-axis, a four-parameter S-curve was fitted and the EC₅₀ value was calculated (FIG. 16). The results showed that the amount of IFN-γ produced by FP-F 1-activated T cells was positively correlated with the antibody concentration and slightly lower than that produced by the control antibody OKT3. This indicated that the anti-CD3 single chain antibody of the bispecific antibody functioned well. The EC₅₀ value that OKT3 activated human PBMCs was 0.015 nM, whereas that of FP-F1 was 0.233 nM. The activation ability of FP-F1 decreased by about two orders of magnitude, which helped to reduce the clinical toxicity of the bispecific antibody.

Another anti-CD3×CD20 bispecific antibody FP-F2 constructed in the present invention also activated T cells and produced IFN-γ in a concentration-dependent manner in the human PBMC cell activation assay. The EC₅₀ that FP-F2 activated human PBMCs was 0.214 nM, which was comparable to that of FP-F1.

7.3 Efficacy of anti-CD3×CD20 Bispecific Antibody FP-F1 in Killing Subcutaneous Xenografts

Human Burkitt's lymphoma Raji Cells (8×10⁶ cells, The Collection Cell Bank of Chinese Academy of Sciences) and Matrigel (BD Biosciences, Cat. No. 354234) were co-inoculated in female SCID Beige mice (Shanghai Lingchang Biotechnology Co., Ltd.) at a ratio of 1:1 subcutaneously. After 6 days of growth, the mice were grouped according to body weight and tumor volume, 7 mice per group. The cultured LAK cells were injected into the tumor tissue at a dose of 1×10⁶ cells/50 μL. Meantime, FP-F1 was administered in the same day at doses of 10, 1 and 0.1 mg/kg, respectively, twice a week by intravenous injection. The control groups were: (1) negative control group: solvent of FP-F1 (PBS); (2) positive control group: Rituxan® (anti-CD20 antibody, Genentech), administered at a dose of 10 mg/kg, twice a week by intravenous injection. The body weight and tumor volume of the mice were measured twice a week. The volume was calculated with ½×length×width×width (mm³).

In FIG. 17, different doses of FP-F1 showed good tumor growth-inhibition. The tumors in 2 mice in the 10 mg/kg group completely disappeared. The tumor in 1 mouse in the 1 mg/kg group had completely disappeared. The 0.1 mg/kg dose administered to one group also showed a certain effect of inhibiting tumor growth. The tumor in the negative control group grew normally and reached 400 mm 3 on the 14th day. The results showed that the therapeutic effect of the 1 mg/kg FP-F1 dose was equivalent to that of the 10 mg/kg Rituxan dose, indicating that the effective dose of the bispecific antibody FP-F1 constructed in the present invention for inhibiting tumor growth in vivo was only 1/10 of that of the anti-CD20 monoclonal antibody (Rituxan®). It was expected that the clinical dose of FP-F 1 would be greatly reduced.

All documents mentioned in the present invention are hereby incorporated by reference to the same extent as if each of the documents is individually recited for reference. It is to be understood that various changes and modifications may be made by those skilled in the art upon reading the above teachings of the present invention, which also fall within the scope of the claims appended hereto.

1.-30. (canceled)

31. A linker peptide for constructing a fusion protein, wherein the linker peptide comprises a flexible peptide and a rigid peptide, wherein:
the flexible peptide comprises one or more flexible units and the rigid peptide comprises one or more rigid units, wherein:
the flexible unit has the structural formula of (GS)a(GGS)b(GGGS)c(GGGGS)d (SEQ ID NO: 37), wherein each of a, b, c, and d is an integer which is equal to or more than 0, and a+b+c+d≥1,
the rigid unit comprises an amino acid sequence having at least 90% or 95% identity to the amino acid sequence of SEQ ID NO: 1 or a fragment thereof.

32. The linker peptide of claim 31, wherein the rigid unit has at least two glycosylation sites.

33. The linker peptide of claim 32, wherein the rigid peptide is located at the C-terminus of the flexible peptide.

34. The linker peptide of claim 33, wherein the rigid peptide comprises 1, 2, 3, 4, or 5 rigid units.

35. The linker peptide of claim 34, wherein the flexible unit comprises an amino acid sequence selected from:
i)
(SEQ ID NO: 21)
GSGGGSGGGGSGGGGS;
ii)
(SEQ ID NO: 22)
GSGGGGSGGGGSGGGGSGGGGSGGGGS;
iii)
(SEQ ID NO: 23)
GSGGGGSGGGGSGGGGSGGGGSGGGGSGGGGSGGGGS;
iv)
(SEQ ID NO: 24)
GSGGGGSGGGGSGGGGS;
v)
(SEQ ID NO: 25)
GGGSGGGSGGGSGGGSGGGS;
and
vi)
(SEQ ID NO: 26)
GGSGGSGGSGGS.

36. The linker peptide of claim 34, wherein the rigid unit comprises an amino acid sequence selected from:
i)
(SEQ ID NO: 27)
SSSSKAPPPSLPSPSRLPGPSDTPILPQ;
ii)
(SEQ ID NO: 28)
PRFQDSSSSKAPPPSLPSPSRLPGPSDTPILPQ;
iii)
(SEQ ID NO: 29)
SSSSKAPPPS;
iv)
(SEQ ID NO: 30)
SRLPGPSDTPILPQ;
and
v)
(SEQ ID NO: 31)
SSSSKAPPPSLPSPSR.

37. The linker peptide of claim 35, wherein the rigid unit comprises an amino acid sequence selected from:
i)
(SEQ ID NO: 27)
SSSSKAPPPSLPSPSRLPGPSDTPILPQ;
ii)
(SEQ ID NO: 28)
PRFQDSSSSKAPPPSLPSPSRLPGPSDTPILPQ;
iii)
(SEQ ID NO: 29)
SSSSKAPPPS;
iv)
(SEQ ID NO: 30)
SRLPGPSDTPILPQ;
and
v)
(SEQ ID NO: 31)
SSSSKAPPPSLPSPSR.

38. The linker peptide of claim 34, wherein the rigid unit comprises an amino acid sequence having at least 90% or 95% identity to any one of SEQ ID NOs:
27, 28, 29, 30, and 31.

39. The linker peptide of claim 35, wherein the rigid unit comprises an amino acid sequence having at least 90% or 95% identity to any one of SEQ ID NOs:
27, 28, 29, 30, and 31.

40. The linker peptide of claim 31, wherein the linker peptide comprises an amino acid sequence of SEQ ID NO: 2.

41. The linker peptide of claim 31, wherein the linker peptide comprises an amino acid sequence of SEQ ID NO: 3.

42. The linker peptide of claim 31, wherein the linker peptide comprises an amino acid sequence of SEQ ID NO: 4.

43. The linker peptide of claim 31, wherein the linker peptide comprises an amino acid sequence of SEQ ID NO: 5.

44. The linker peptide of claim 31, wherein the linker peptide comprises an amino acid sequence of SEQ ID NO: 6.

45. The linker peptide of claim 31, wherein the linker peptide comprises an amino acid sequence of SEQ ID NO: 7.


SEQUENCE LISTING (38 sequences)

SEQ ID NO: 1 - protein, 33 aa, Homo sapiens
PRFQDSSSSKAPPPSLPSPSRLPGPSDTPILPQ

SEQ ID NO: 2 - protein, 55 aa, synthetic construct
  REGION 1..55: note = Description of Artificial Sequence: Synthetic polypeptide
  REGION 1..55: note = Amino Acid Sequence of Peptide Linker F2-R1
GSGGGGSGGGGSGGGGSGGGGSGGGGSSSSSKAPPPSLPSPSRLPGPSDTPILPQ

SEQ ID NO: 3 - protein, 49 aa, synthetic construct
  REGION 1..49: note = Description of Artificial Sequence: Synthetic polypeptide
  REGION 1..49: note = Amino Acid Sequence of Peptide Linker F1-R2
GSGGGSGGGGSGGGGSPRFQDSSSSKAPPPSLPSPSRLPGPSDTPILPQ

SEQ ID NO: 4 - protein, 45 aa, synthetic construct
  REGION 1..45: note = Description of Artificial Sequence: Synthetic polypeptide
  REGION 1..45: note = Amino Acid Sequence of Peptide Linker F4-R1
GSGGGGSGGGGSGGGGSSSSSKAPPPSLPSPSRLPGPSDTPILPQ

SEQ ID NO: 5 - protein, 36 aa, synthetic construct
  REGION 1..36: note = Description of Artificial Sequence: Synthetic polypeptide
  REGION 1..36: note = Amino Acid Sequence of Peptide Linker F5-R5
GGGSGGGSGGGSGGGSGGGSSSSSKAPPPSLPSPSR

SEQ ID NO: 6 - protein, 37 aa, synthetic construct
  REGION 1..37: note = Description of Artificial Sequence: Synthetic polypeptide
  REGION 1..37: note = Amino Acid Sequence of Peptide Linker F4-R3R3
GSGGGGSGGGGSGGGGSSSSSKAPPPSSSSSKAPPPS

SEQ ID NO: 7 - protein, 54 aa, synthetic construct
  REGION 1..54: note = Description of Artificial Sequence: Synthetic polypeptide
  REGION 1..54: note = Amino Acid Sequence of Peptide Linker F6-R4R4R4
GGSGGSGGSGGSSRLPGPSDTPILPQSRLPGPSDTPILPQSRLPGPSDTPILPQ

SEQ ID NO: 8 - protein, 227 aa, synthetic construct
  REGION 1..227: note = Description of Artificial Sequence: Synthetic polypeptide
  REGION 1..227: note = Amino Acid Sequence of vFc gamma 1
DKTHTCPPCPAPEVAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 223 aa, synthetic construct
  REGION 1..223: note = Description of Artificial Sequence: Synthetic polypeptide
  REGION 1..223: note = Amino Acid Sequence of vFc gamma 2-1
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 223 aa, synthetic construct
  REGION 1..223: note = Description of Artificial Sequence: Synthetic polypeptide
  REGION 1..223: note = Amino Acid Sequence of vFc gamma 2-2
VECPPCPAPPVAGPSVFLFPPKPKDQLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPGK

SEQ ID NO: 11 - protein, 223 aa, synthetic construct
  REGION 1..223: note = Description of Artificial Sequence: Synthetic polypeptide
  REGION 1..223: note = Amino Acid Sequence of vFc gamma 2-3
VECPPCPAPPVAGPSVFLFPPKPKDQLMISRTPEVTCVVVDVSHEDPEVQFNWYVDGVEVHNAKTKPREEQFNSTFRVVSVLTVVHQDWLNGKEYKCKVSNKGLPASIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVLHEALHNHYTQKSLSLSPGK

SEQ ID NO: 12 - protein, 229 aa, synthetic construct
  REGION 1..229: note = Description of Artificial Sequence: Synthetic polypeptide
  REGION 1..229: note = Amino Acid Sequence of vFc gamma 4
ESKYGPPCPPCPAPEFAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 406 aa, synthetic construct [sequence omitted]

SEQ ID NO: 14 - protein, 1438 aa, synthetic construct
  REGION 1..1438: note = Description of Artificial Sequence: Synthetic polypeptide
  REGION 1..1438: note = Amino Acid Sequence of Mature FVIII Protein
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 15 - protein, 39 aa, Heloderma suspectum
HGEGTFTSDLSKQMEEEAVRLFIEWLKNGGPSSGAPPPS

SEQ ID NO: 16 - protein, 152 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 17 - protein, 191 aa, Homo sapiens [sequence omitted]

SEQ ID NO: 18 - protein, 451 aa, synthetic construct
  REGION 1..451: note = Description of Artificial Sequence: Synthetic polypeptide
  REGION 1..451: note = Amino Acid Sequence of the Heavy Chain of Anti-CD20 Antibody
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 214 aa, synthetic construct
  REGION 1..214: note = Description of Artificial Sequence: Synthetic polypeptide
  REGION 1..214: note = Amino Acid Sequence of the Light Chain of Anti-CD20 antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 241 aa, synthetic construct
  REGION 1..241: note = Description of Artificial Sequence: Synthetic polypeptide
  REGION 1..241: note = Amino Acid Sequence of Anti-CD3 scFv
QVQLVQSGGGVVQPGRSLRLSCKASGYTFTRYTMHWVRQAPGKGLEWIGYINPSRGYTNYNQKVKDRFTISTDKSKSTAFLQMDSLRPEDTAVYYCARYYDDHYCLDYWGQGTTLTVSSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITR

SEQ ID NO: 21 - protein, 16 aa, synthetic construct
  REGION 1..16: note = Description of Artificial Sequence: Synthetic peptide
  REGION 1..16: note = Amino Acid Sequence of flexible unit F1
GSGGGSGGGGSGGGGS

SEQ ID NO: 22 - protein, 27 aa, synthetic construct
  REGION 1..27: note = Description of Artificial Sequence: Synthetic peptide
  REGION 1..27: note = Amino Acid Sequence of flexible unit F2
GSGGGGSGGGGSGGGGSGGGGSGGGGS

SEQ ID NO: 23 - protein, 37 aa, synthetic construct
  REGION 1..37: note = Description of Artificial Sequence: Synthetic polypeptide
  REGION 1..37: note = Amino Acid Sequence of flexible unit F3
GSGGGGSGGGGSGGGGSGGGGSGGGGSGGGGSGGGGS

SEQ ID NO: 24 - protein, 17 aa, synthetic construct
  REGION 1..17: note = Description of Artificial Sequence: Synthetic peptide
  REGION 1..17: note = Amino Acid Sequence of Flexible Unit F4
GSGGGGSGGGGSGGGGS

SEQ ID NO: 25 - protein, 20 aa, synthetic construct
  REGION 1..20: note = Description of Artificial Sequence: Synthetic peptide
  REGION 1..20: note = Amino Acid Sequence of Flexibile Unit F5
GGGSGGGSGGGSGGGSGGGS

SEQ ID NO: 26 - protein, 12 aa, synthetic construct
  REGION 1..12: note = Description of Artificial Sequence: Synthetic peptide
  REGION 1..12: note = Amino Acid Sequence of Flexible Unit F6
GGSGGSGGSGGS

SEQ ID NO: 27 - protein, 28 aa, synthetic construct
  REGION 1..28: note = Description of Artificial Sequence: Synthetic peptide
  REGION 1..28: note = Amino Acid Sequence of Rigid Unit R1
SSSSKAPPPSLPSPSRLPGPSDTPILPQ

SEQ ID NO: 28 - protein, 33 aa, synthetic construct; identical to SEQ ID NO: 1
  REGION 1..33: note = Description of Artificial Sequence: Synthetic polypeptide
  REGION 1..33: note = Amino Acid Sequence of Rigid Unit R2

SEQ ID NO: 29 - protein, 10 aa, synthetic construct
  REGION 1..10: note = Description of Artificial Sequence: Synthetic peptide
  REGION 1..10: note = Amino Acid Sequence of Rigid Unit R3
SSSSKAPPPS

SEQ ID NO: 30 - protein, 14 aa, synthetic construct
  REGION 1..14: note = Description of Artificial Sequence: Synthetic peptide
  REGION 1..14: note = Amino Acid Sequence of Rigid Unit R4
SRLPGPSDTPILPQ

SEQ ID NO: 31 - protein, 16 aa, synthetic construct
  REGION 1..16: note = Description of Artificial Sequence: Synthetic peptide
  REGION 1..16: note = Amino Acid Sequence of Rigid Unit R5
SSSSKAPPPSLPSPSR

SEQ ID NO: 32 - protein, 20 aa, synthetic construct
  REGION 1..20: note = Description of Artificial Sequence: Synthetic peptide
  REGION 1..20: note = Amino Acid Sequence of Rigid Peptide R3-R3
SSSSKAPPPSSSSSKAPPPS

SEQ ID NO: 33 - protein, 42 aa, synthetic construct
  REGION 1..42: note = Description of Artificial Sequence: Synthetic polypeptide
  REGION 1..42: note = Amino Acid Sequence of Rigid Peptide R4-R4-R4
SRLPGPSDTPILPQSRLPGPSDTPILPQSRLPGPSDTPILPQ

SEQ ID NO: 34 - protein, 7 aa, synthetic construct
  REGION 1..7: note = Description of Artificial Sequence: Synthetic peptide
AEAAAKA

SEQ ID NO: 35 - protein, 15 aa, synthetic construct
  REGION 1..15: note = Description of Artificial Sequence: Synthetic peptide
GGGGSGGGGSGGGGS

SEQ ID NO: 36 - protein, 14 aa, synthetic construct
  REGION 1..14: note = Description of Artificial Sequence: Synthetic peptide
GSTSGSGKSSEGKG

SEQ ID NO: 37 - protein, 14 aa, synthetic construct
  REGION 1..14: note = Description of Artificial Sequence: Synthetic peptide
  VARIANT 1..2: note = May or may not be present
  VARIANT 3..5: note = May or may not be present
  VARIANT 6..9: note = May or may not be present
  VARIANT 10..14: note = May or may not be present
  REGION 1..14: note = See specification as filed for detailed description of substitutions and preferred embodiments
GSGGSGGGSGGGGS

SEQ ID NO: 38 - protein, 5 aa, synthetic construct
  REGION 1..5: note = Description of Artificial Sequence: Synthetic peptide
GGGGS